(19) United States
(12) Patent Application Publication
(10) Pub. No.: US 2011/0213028 A1
(43) Pub. Date: Sep. 1, 2011

(54) FATTY ACID MYCOPHENOLATE DERIVATIVES AND THEIR USES

(75) Inventors: Jill C. Milne, Brookline, MA (US); Michael R. Jirousek, Cambridge, MA (US); Jean E. Bemis, Arlington, MA (US); Chi B. Vu, Arlington, MA (US)

(73) Assignee: CATABASIS PHARMACEUTICALS, INC., Cambridge, MA (US)

(21) Appl. No.: 13/035,622

(22) Filed: Feb. 25, 2011

Related U.S. Application Data
(60) Provisional application No. 61/308,552, filed on Feb. 26, 2010.

(51) Int. Cl. A61K 31/365; C07D 307/88; A61P 37/06; A61P 17/06; A61P 25/00; A61P 17/00

(52) U.S. Cl. 514/470; 549/310

(57) ABSTRACT

The invention relates to fatty acid mycophenolate derivatives; compositions comprising an effective amount of a fatty acid mycophenolate derivative; and methods for treating and preventing organ rejection and autoimmune diseases such as systemic lupus erythematosus, psoriasis and multiple sclerosis comprising the administration of an effective amount of a fatty acid mycophenolate derivative.

The present application claims the benefit of U.S. Provisional Application No. 61/308,552 filed Feb. 26, 2010, the entire disclosure of which is relied on for all purposes and is incorported into this application by reference.

### FIELD OF THE INVENTION

The invention relates to fatty acid mycophenolate derivatives; compositions comprising an effective amount of a fatty acid mycophenolate derivative; and methods for treating or preventing a metabolic, autoimmune or neurodegenerative disorder comprising the administration of an effective amount of a fatty acid mycophenolate derivative. All patents, patent applications, and publications cited herein are hereby incorporated by reference in their entireties.

### BACKGROUND OF THE INVENTION

Oily cold water fish, such as salmon, trout, herring, and tuna are the source of dietary marine omega-3 fatty acids, with eicosapentaenoic acid (EPA) and docosahexaenoic acid (DHA) being the key marine derived omega-3 fatty acids. Omega-3 fatty acids have previously been shown to improve insulin sensitivity and glucose tolerance in normoglycemic men and in obese individuals. Omega-3 fatty acids have also been shown to improve insulin resistance in obese and non-obese patients with an inflammatory phenotype. Lipid, glucose, and insulin metabolism have been shown to improve in overweight hypertensive subjects through treatment with omega-3 fatty acids. Omega-3 fatty acids (EPA/DHA) have also been shown to decrease triglycerides and to reduce the risk for sudden death caused by cardiac arrhythmias in addition to improve mortality in patients at risk of a cardiovascular event. Omega-3 fatty acids have also been taken as the dietary supplement portion of therapy used to treat dyslipidemia. Last, but not least, omega-3 fatty acids have been known to have a number of anti-inflammatory properties. A higher intake of omega-3 fatty acids lower levels of circulating TNF-α and IL-6, two of the cytokines that are markedly increased during inflammation processes (Chapkin et al, Prostaglandins, Leukot Essent Fatty Acids 2009, 81, p. 187-191; Duda et al, Cardiovasc Res 2009, 84, p. 33-41). In addition, a higher intake of omega-3 fatty acids has been shown to increase levels of the well-characterized anti-inflammatory cytokine IL-10 (Bradley et al, Obesity (Silver Spring) 2008, 16, p. 938-944). In a 24 week randomized double blind placebo controlled clinical trial, involving 60 patients with systemic lupus erythematosus, 3 grams of omega-3 fatty acids was given a day. At the end of the 24 week period, a significant improvement in a number of parameters was observed: SLAM-R score (revised Systemic Lupus Activity Measurement), BILAG score (British Isles Lupus Assessment Group), FMD (flow-mediated dilation) and platelet 8-isoprostanes (Wright, S. A. et al. Ann. Rheum. Dis. 2008, 67 (6), 841-848).

Both DHA and EPA are characterized as long chain fatty acids (aliphatic portion between 12-22 carbons). Medium chain fatty acids are characterized as those having the aliphatic portion between 6-12 carbons. Lipoic acid is a medium chain fatty acid found naturally in the body. It plays many important roles such as free radical scavenger, chelator to heavy metals and signal transduction mediator in various inflammatory and metabolic pathways, including the NF-κB pathway (Shay, K. P. et al. Biochim. Biophys. Acta 2009, 1790, 1149-1160). Lipoic acid has been found to be useful in a number of chronic diseases that are associated with oxidative stress (for a review see Smith, A. R. et al Curr. Med. Chem. 2004, 11, p. 1135-46). Lipoic acid has now been evaluated in the clinic for the treatment of diabetes (Morcos, M. et al Diabetes Res. Clin. Pract. 2001, 52, p. 175-183) and diabetic neuropathy (Mijnhout, G. S. et al Neth. J. Med. 2010, 110, p. 158-162). Lipoic acid has also been found to be potentially useful in treating cardiovascular diseases (Ghibu, S. et al, J. Cardiovasc. Pharmacol. 2009, 54, p. 391-8), Alzheimer's disease (Maczurek, A. et al, Adv. Drug Deliv. Rev. 2008, 60, p. 1463-70) and multiple sclerosis (Yadav, V. Multiple Sclerosis 2005, 11, p. 159-65; Salinthone, S. et al, Endocr. Metab. Immune Disord. Drug Targets 2008, 8, p. 132-42).

Mycophenolate mofetil is the mofetil prodrug ester of mycophenolic acid, an effective immunosuppressive agent that has been approved by the FDA for preventing and reducing the frequency and severity of acute rejection episodes in kidney, heart and liver transplants. Mycophenolic acid is a selective, reversible and non-competitive inhibitor of inosine monophosphate dehydrogenase, a critical enzyme for the de novo synthesis of guanosine nucleotides. By blocking the production of guanosine nucleotides, mycophenolic acid is able to effectively inhibit T and B cell proliferation. Mycophenolate mofetil has also been shown to be effective in different models of systemic lupus erythematosus. In the female (NZB X NZW)F1 mouse model of systemic lupus erythematosus, mycophenolate mofetil was able to suppress autoimmunity and mortality when given 200 mg/kg/day over a 6 week period (Elbourne, K.B. et al. J. Rheum. 1998, 25(12), 2364-2370). In the SLE-prone MRLlpr/lpr mice, mycophenolate mofetil (at 100 mg/kg in drinking water) was able to prolong survival, reduce the extent of deposition of C3 in glomeruli and reduce the occurrence of albuminuria and haematuria (Jonsson et al. Clin. Exp. Immunol. 1999, 116 (3), 534-541). In randomized clinical trials involving 847 patients with proliferative lupus nephritis, mycophenolate mofetil offered similar efficacy in renal remission and survival as cyclophosphamide (Mak, A. et al. Rheumatology 2009, 48 (8), 944-952). Mycophenolate mofetil is also effective in reducing disease flares in systemic lupus erythematosus patients when administered at a mean dose of 1,328 mg/a day over a 1 or 2 year period (Nannini, C. et al. Lupus 2009, 18 (5), 394-399).

The ability to provide the effects of fatty acid and mycophenolate in a synergistic way would provide benefits in preventing and reducing the frequency and severity of acute rejection episodes in organ transplants and for treating systemic lupus erythematosus and other autoimmune diseases.

### SUMMARY OF THE INVENTION

The invention is based in part on the discovery of fatty acid mycophenolate derivatives and their demonstrated effects in achieving improved treatment that cannot be achieved by administering mycophenolate or fatty acids alone or in combination. These novel compounds are useful in preventing and reducing the frequency and severity of acute rejection episodes in organ transplants; reducing, inhibiting or treating restenosis and hyperplasia; hyperproliferative vascular disease; angiogenesis and for treating systemic lupus erythematosus, psoriasis, and other autoimmune diseases.

Accordingly in one aspect, a molecular conjugate is described which comprises a mycophenolate and a fatty acid covalently linked, wherein the fatty acid is selected from the group consisting of omega-3 fatty acids, fatty acids that are metabolized in vivo to omega-3 fatty acids, and lipoic acid, and the conjugate is capable of hydrolysis to produce free mycophenolate and free fatty acid. In some embodiments, the fatty acid is selected from the group consisting of all-cis-7,10,13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid and lipoic acid. In other embodiments, the fatty acid is selected from eicosapentaenoic acid, docosahexaenoic acid and lipoic acid. In some embodiments, the hydrolysis is enzymatic.

Accordingly, in one aspect, compounds of the Formula I are described:

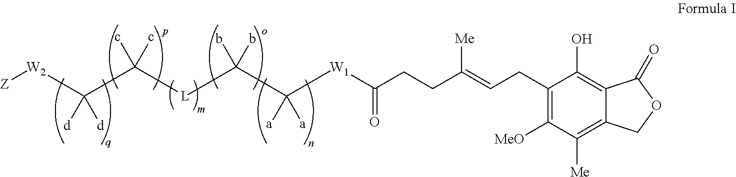

and pharmaceutically acceptable salts, hydrates, solvates, prodrugs, enantiomers, and stereoisomers thereof;

wherein

W₁ and W₂ are each independently null, O, S, NH, NR, or W₁ and W₂ can be taken together can form an imidazolidine or piperazine group,

each a, b, c, and d is independently —H, -D, —CH₃, —OCH₃, —OCH₂CH₃, —C(O)OR, —O-Z, or benzyl, or two of a, b, c, and d can be taken together, along with the single carbon to which they are bound, to form a cycloalkyl or heterocycle;

each n, o, p, and q is independently 0, 1 or 2;

L is independently null, —O—, —S—, —S(O)—, —S(O)₂—, —S—S—, —(C₁-C₆alkyl)—, —(C₃-C₆cycloalkyl)-, a heterocycle, a heteroaryl,

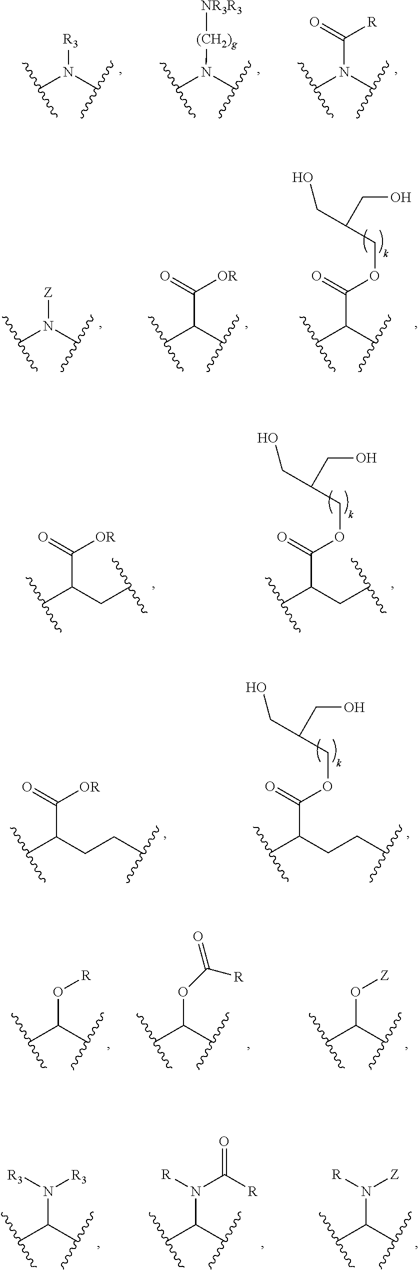

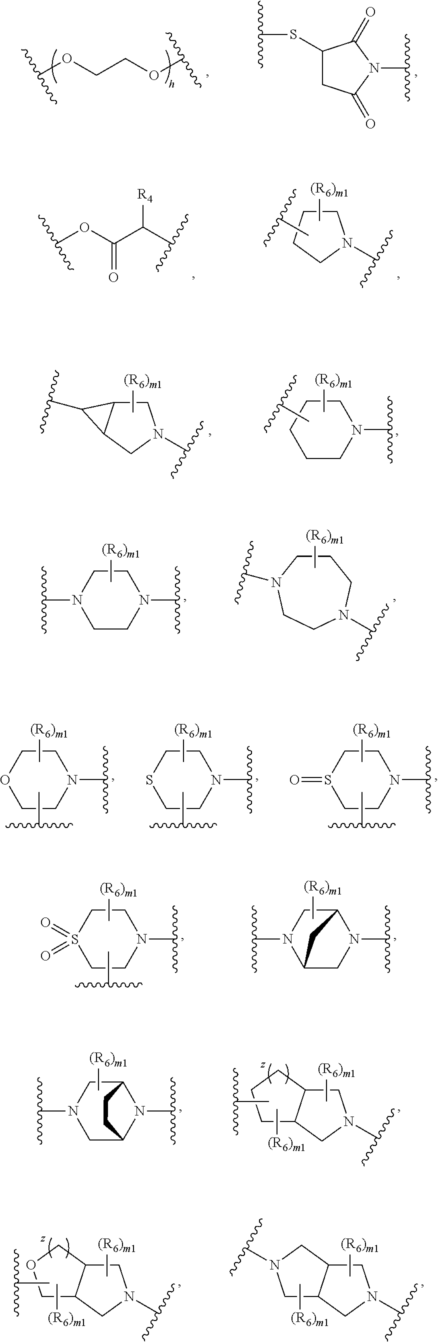

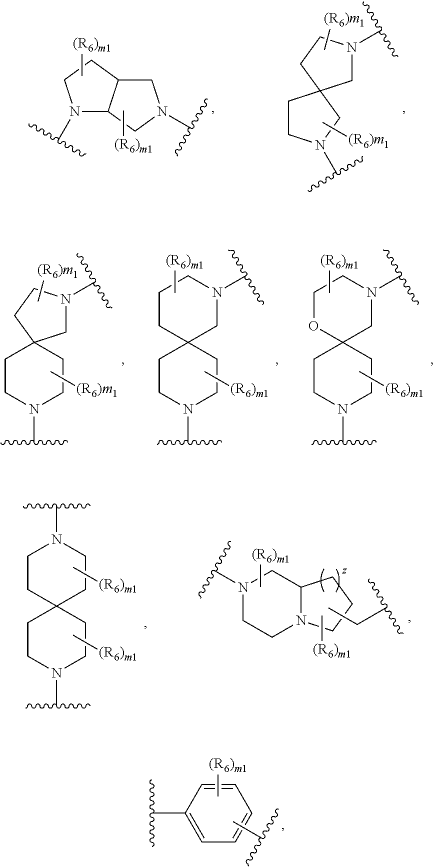

wherein the representation of L is not limited directionally left to right as is depicted, rather either the left side or the right side of L can be bound to the W₁ side of the compound of Formula I;

R₆ is independently —H, -D, -C₁—C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;

each g is independently 2, 3 or 4;

each h is independently 1, 2, 3 or 4;

m is 0, 1, 2, or 3; if m is more than 1, then L can be the same or different;

m1 is 0, 1, 2 or 3;

k is 0, 1, 2, or 3;

z is 1, 2, or 3;

each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;

each R₄ is independently e, H or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each Z is independently —H,

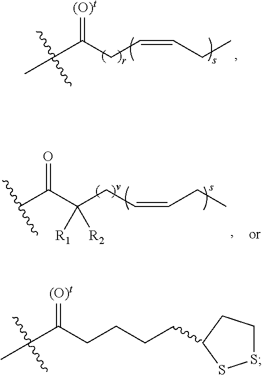

with the proviso that there is at least one

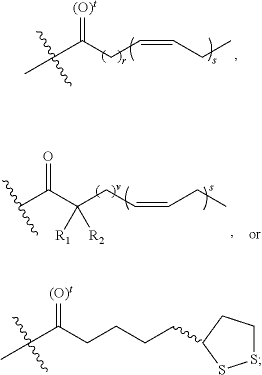

in the compound;

each r is independently 2, 3, or 7;

each s is independently 3, 5, or 6;

each t is independently 0 or 1;

each v is independently 1, 2, or 6;

R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, -C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and

each R is independently —H, —C₁-C₃ alkyl, or straight or branched C₁-C₄ alkyl optionally substituted with OH, or halogen;

provided that
when m, n, o, p, and q are each 0, W₁ and W₂ are each null, and Z is

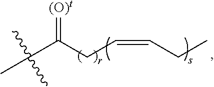

then t must be 0; and
when each of m, n, o, p, and q is 0, and W₁ and W₂ are each null, then Z must not be

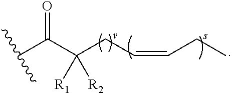

In Formula I, any one or more of H may be substituted with a deuterium. It is also understood in Formula I, that a methyl substituent can be substituted with a C₁-C₆ alkyl.

Also described are pharmaceutical formulations comprising at least one fatty acid mycophenolate derivative.

Also described herein are methods of treating a disease susceptible to treatment with a fatty acid mycophenolate derivative in a patient in need thereof by administering to the patient an effective amount of a fatty acid mycophenolate derivative.

Also described herein are methods of treating metabolic diseases or autoimmune disease or neurodegenerative diseases by administering to a patient in need thereof an effective amount of a fatty acid mycophenolate derivative.

The invention also includes pharmaceutical compositions that comprise an effective amount of a fatty acid mycophenolate derivative and a pharmaceutically acceptable carrier. The compositions are useful for preventing and reducing the frequency and severity of acute rejection episodes in organ transplants and for treating systemic lupus erythematosus and other autoimmune diseases. The invention includes a fatty acid mycophenolate derivative provided as a pharmaceutically acceptable prodrug, a hydrate, a salt, such as a pharmaceutically acceptable salt, enantiomer, stereoisomer, or mixtures thereof.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### BRIEF DESCRIPTION OF THE FIGURES

FIG. 1 depicts the effects of compounds I-1, I-2, I-5, and I-10 on the expression of IL-1β in RAW 264.7 macrophages.

### DETAILED DESCRIPTION OF THE INVENTION

As described earlier, omega-3 fatty acids such as DHA and EPA, and the fatty acid lipoic acid have been known to have anti-inflammatory properties. Mycophenolate has been used as an immunosuppressive agent to prevent and reduce the frequency of organ rejection and as an anti-proliferative agent to treat systemic lupus erythematosus. The fatty acid mycophenolate derivatives can prevent and reduce the frequency and severity of acute rejection episodes in organ transplants or treat systemic lupus erythematosus and other autoimmune diseases.

The fatty acid mycophenolate derivatives have been designed to bring together mycophenolic acid and fatty acids into a single molecular conjugate. The activity of the fatty acid mycophenolate derivatives is substantially greater than the sum of the individual components of the molecular conjugate, suggesting that the activity induced by the fatty acid mycophenolate derivatives is synergistic.

### DEFINITIONS

The following definitions are used in connection with the fatty acid mycophenolate derivatives:

The term “fatty acid mycophenolate derivatives” includes any and all possible isomers, stereoisomers, enantiomers, diastereomers, tautomers, pharmaceutically acceptable salts, hydrates, solvates, and prodrugs of the fatty acid mycophenolate derivatives described herein.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl and cyclohexyl. It is understood that any of the substitutable hydrogens on a cycloalkyl can be substituted with halogen, C₁-C₃ alkyl, hydroxyl, alkoxy and cyano groups.

The term “heterocycle” as used herein refers to a cyclic hydrocarbon containing 3-6 atoms wherein at least one of the atoms is an O, N, or S. Examples of heterocycles include, but are not limited to, aziridine, oxirane, thiirane, azetidine, oxetane, thietane, pyrrolidine, tetrahydrofuran, tetrahydrothiophene, piperidine, tetrahydropyran, thiane, imidazolidine, oxazolidine, thiazolidine, dioxolane, dithiolane, piperazine, oxazine, dithiane, and dioxane.

The term “heteroaryl” as used herein refers to a monocyclic or bicyclic ring structure having 5 to 12 ring atoms wherein one or more of the ring atoms is a heteroatom, e.g. N, O or S and wherein one or more rings of the bicyclic ring structure is aromatic. Some examples of heteroaryl are pyridyl, furyl, pyrrolyl, thienyl, thiazolyl, oxazolyl, imidazolyl, indolyl, tetrazolyl, benzofuryl, xanthenes and dihydroindole. It is understood that any of the substitutable hydrogens on a heteroaryl can be substituted with halogen, C₁-C₃ alkyl, hydroxyl, alkoxy and cyano groups.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

The term “fatty acid” as used herein means an omega-3 fatty acid and fatty acids that are metabolized in vivo to omega-3 fatty acids. Non-limiting examples of fatty acids are all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis-5,8,11,14,17-eicosapentaenoic acid), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21-docosahexaenoic acid), or tetracosahexaenoic acid (nisinic acid or all-cis-6,9,12,15,18,21-tetracosenoic acid). In addition, the term “fatty acid” can also refer to medium chain fatty acids such as lipoic acid.

The term “mycophenolic acid” as used herein means the molecule known as mycophenolic acid and any derivative thereof.

The term “mycophenolate” as used herein means esters and salts of the molecule known as mycophenolic acid and any derivative thereof.

A “subject” is a mammal, e.g., a human, mouse, rat, guinea pig, dog, cat, horse, cow, pig, or non-human primate, such as a monkey, chimpanzee, baboon or rhesus, and the terms “subject” and “patient” are used interchangeably herein.

The invention also includes pharmaceutical compositions comprising an effective amount of a fatty acid mycophenolate derivative and a pharmaceutically acceptable carrier. The invention includes a fatty acid mycophenolate derivative provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2,2 -disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “metabolic disease” as used herein refers to disorders, diseases and syndromes involving dyslipidemia, and the terms metabolic disorder, metabolic disease, and metabolic syndrome are used interchangeably herein.

An “effective amount” when used in connection with a fatty acid mycophenolate derivative is an amount effective for treating or preventing a metabolic disease.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating”, with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a fatty acid mycophenolate derivative.

The following abbreviations are used herein and have the indicated definitions: Boc and BOC are tert-butoxycarbonyl, Boc₂O is di-tert-butyl dicarbonate, CDI is 1,1′-carbonyldiimidazole, DCC is N,N′-dicyclohexylcarbodiimide, DIEA is N,N-diisopropylethylamine, DMAP is 4-dimethylaminopyridine, DNA is deoxyribonucleic acid DOSS is sodium dioctyl sulfosuccinate, EDC and EDCI are 1-ethyl-3-(3-dimethylaminopropyl)carbodiimide hydrochloride, ELISA is enzyme-linked immunosorbent assay, EtOAc is ethyl acetate, eq is equivalents, FITC is fluorescein isothiocyanate, FDA is Food and Drug Administration, h is hour, HATU is 2-(7-aza-1H-benzotriazole-1-yl)-1,1,3,3-tetramethyluronium hexafluorophosphate, HPMC is hydroxypropyl methylcellulose, oxone is potassium peroxymonosulfate, Pd/C is palladium on carbon, TFA is trifluoroacetic acid, TGPS is tocopherol propylene glycol succinate, THF is tetrahydrofuran, and TNF is tumor necrosis factor.

Accordingly in one aspect, a molecular conjugate is described which comprises a mycophenolate and a fatty acid covalently linked, wherein the fatty acid is selected from the group consisting of omega-3 fatty acids, fatty acids that are metabolized in vivo to omega-3 fatty acids, and lipoic acid, and the conjugate is capable of hydrolysis to produce free mycophenolate and free fatty acid.

In some embodiments, the fatty acid is selected from the group consisting of all-cis-7,10,13-hexadecatrienoic acid, a-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid, and lipoic acid. In other embodiments, the fatty acid is selected from eicosapentaenoic acid and docosahexaenoic acid. In some embodiments, the fatty acid is lipoic acid. In some embodiments, the hydrolysis is enzymatic.

In another aspect, the present invention provides fatty acid mycophenolate derivatives according to Formula I:

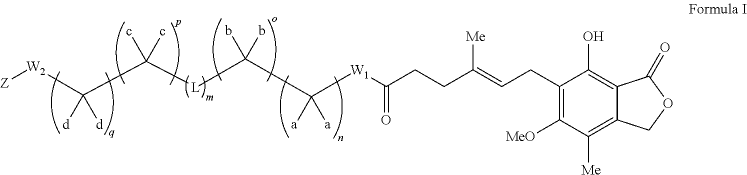

and pharmaceutically acceptable salts, hydrates, solvates, prodrugs, enantiomers and stereoisomers thereof;

wherein

W₁, W_{2 9} a, b, c, d, e, k, m, n, o, p, q, L, Z, r, s, t, v, z, R₁, R₂, R₃, R₄, R and R₆ are as defined above for Formula I,

with the proviso that there is at least one

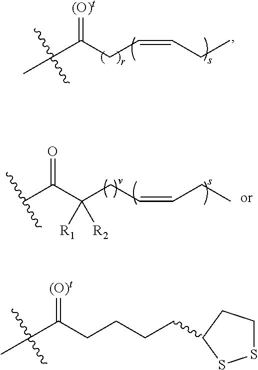

in the compound.

In some embodiments, one Z is

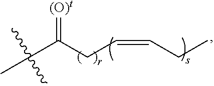

and r is 2.

In some embodiments, one Z is

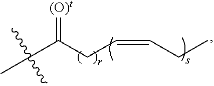

and r is 3.

In some embodiments, one Z is

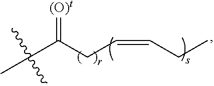

and r is 7.

In other embodiments, one Z is

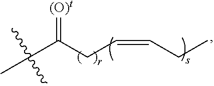

and s is 3.

In some embodiments, one Z is

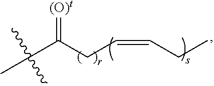

and s is 5.

In some embodiments, one Z is

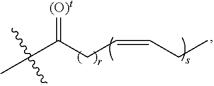

and s is 6.

In some embodiments, one Z is

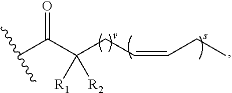

and v is 1.

In other embodiments, one Z is

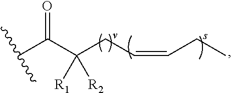

and v is 2.

In some embodiments, one Z is

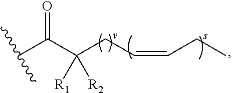

and v is 6.

In some embodiments, one Z is

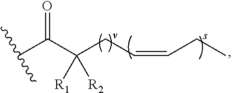

and s is 3.

In some embodiments, one Z is

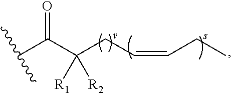

and s is 5.

In other embodiments, one Z is

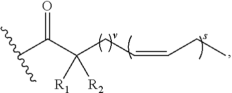

and s is 6.

In other embodiments, Z is

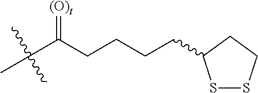

and t is 1.

In some embodiments, Z is

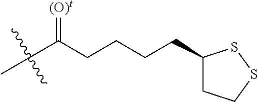

and t is 1.

In some embodiments, W₁ is NH.

In some embodiments, W₂ is NH.

In some embodiments, W₁ is O.

In some embodiments, W₂ is O.

In some embodiments, W₁ is null

In some embodiments, W₂ is null

In some embodiments, W₁ and W₂ are each NH.

In some embodiments, W₁ and W₂ are each null

In some embodiments, W₁ is O and W₂ is NH.

In some embodiments, W₁ and W₂ are each NR, and R is CH₃.

In some embodiments, m is 0.

In other embodiments, m is 1.

In other embodiments, m is 2.

In some embodiments, L is —S— or —S—S—.

In some embodiments, L is —O—.

In some embodiments, L is —C(O)—.

In some embodiments, L is heteroaryl.

In some embodiments, L is heterocycle.

In some embodiments, L is

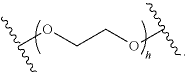

In some embodiments, L is

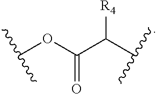

In some embodiments, L is

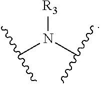

In some embodiments, L is

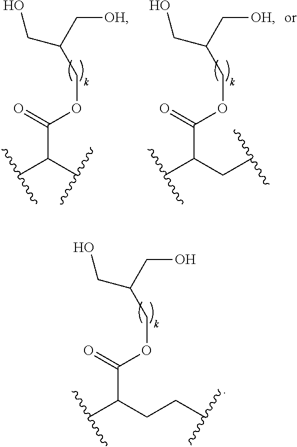

In some embodiments, L is

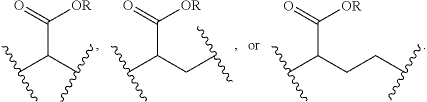

In some embodiments, L is

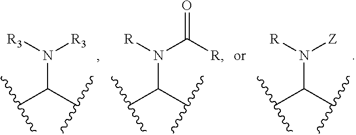

In some embodiments, L is

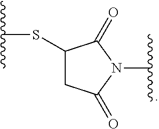

In some embodiments, L is

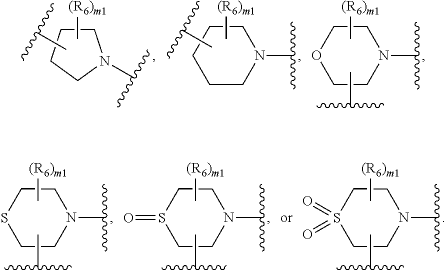

In some embodiments, L is

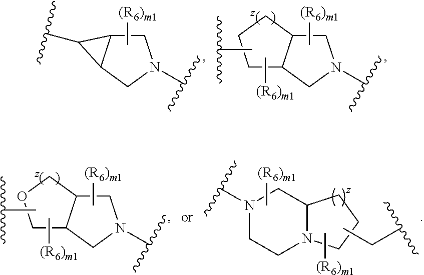

In some embodiments, L is

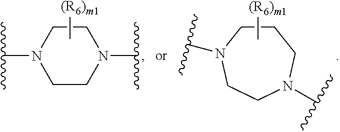

In some embodiments, L is

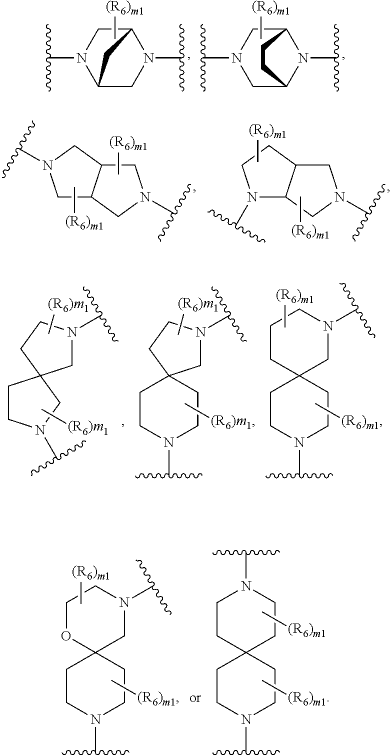

In other embodiments, one of n, o, p, and q is 1.

In some embodiments, two of n, o, p, and q are each 1.

In other embodiments, three of n, o, p, and q are each 1.

In some embodiments n, o, p, and q are each 1.

In some embodiments, one d is C(O)OR.

In some embodiments, r is 2 and s is 6.

In some embodiments, r is 3 and s is 5.

In some embodiments, t is 1.

In some embodiments, W₁ and W₂ are each NH, m is 0, n, and o are each 1, and p and q are each 0.

In some embodiments, W₁ and W₂ are each NH, m is 1, n, o, p, and q are each 1, and L is O.

In some embodiments, W₁ and W₂ are each NH, m is 1, n, o, p, and q are each 1, and L is

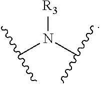

In some embodiments, W₁ and W₂ are each NH, m is 1, n, o, p, and q are each 1, and L is —S—S—.

In some embodiments, W₁ and W₂ are each NH, m is 1, n and o are each 0, p and q are each 1, and L is

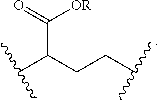

In some embodiments, W₁ and W₂ are each NH, m is 1, k is O, n and o are each 0, p and q are each 1, and L is

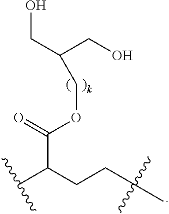

In some embodiments, W₁ and W₂ are each NH, m is 1, n and o are each 1, p and q are each 0, and L is

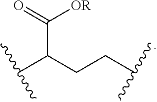

In some embodiments, W₁ and W₂ are each NH, m is 1, k is 0, n is 1, o, p and q are each 0, and L is

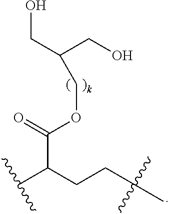

In some embodiments, W₁ and W₂ are each NH, m is 1, n, o, and p are each 0, and q is 1, and L is

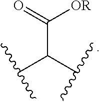

In some embodiments, W₁ and W₂ are each NH, m is 1, k is 1, n, o, and p are each 0, and q is 1, and L is

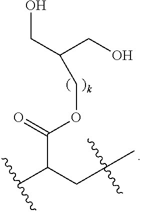

In some embodiments, W₁ and W₂ are each NH, m is 1, n is 1, and o, p, and q are each 0, and L is

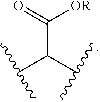

In some embodiments, W₁ and W₂ are each NH, m is 1, k is 1, o, p, and q are each 0, and L is

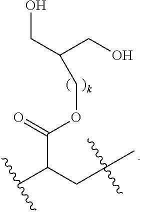

In some embodiments, W₁ and W₂ are each NH, m is 1, n, o, p, and q are each 1, and L is

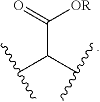

In some embodiments, W₁ and W₂ are each NH, m is 1, n, o, p, and q are each 1, and L is

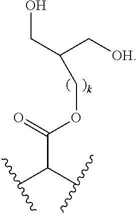

In some embodiments, W₁ and W₂ are each NH, m is 0, k is 1, o and p are each 1, and q is 0.

In some embodiments, W₁ and W₂ are each NH, m is 0, n, o, p, and q are each 1.

In some embodiments, W₁ and W₂ are each NH, m is 0, n and o are each 1, p and q are each 0, and each a is CH₃.

In some embodiments, W₁ and W₂ are each NH, m is 0, n and o are each 1, p and q are each 0, and each b is CH₃.

In some embodiments, W₁ and W₂ are each NH, m is 1, n, o, p, and q are each 1, R₃ is H, and L is

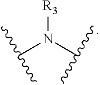

In some embodiments, W₁ and W₂ are each NH, m is 1, n, p and q are each 1, and o is 2, R₃ is H, and L is

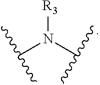

In some embodiments, W₁ and W₂ are each NH, m is 1, n, o, p are each 1, and q is 2, and L is

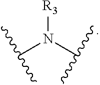

In some embodiments, W₁ and W₂ are each NH, m is 1, n, o, p, and q are each 1, and L is

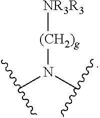

In some embodiments, W₁ and W₂ are each NH, m is 1, n and p are each 1, and o and q are each 0, and L is —C(O)—.

In some embodiments, W₁ and W₂ are each NH, m is 1, n and p are each 1, and o, and q are each 0, and L is

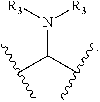

In some embodiments, W₁ and W₂ are each NH, m is 1, n, o, p, q are each 1, and

L is

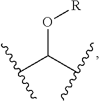

In some embodiments, W₁ and W₂ are each NH, m is 1, n, o, p , and q are each 1, h is 1, and L is

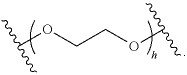

In some embodiments, W₁ and W₂ are each NH, m is 1, n, o, p , and q are each 1, and L is —S—.

In some embodiments, W₁ and W₂ are each NH, m is 1, n, o, p are each 0, q is 1, one d is —CH₃, and L is

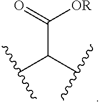

In some embodiments, W₁ and W₂ are each NH, m is 2, n, o, p, and q are each 0, one L is

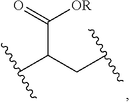

and

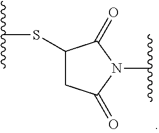

### one L is

In some embodiments, m is 0, n, o, p, and q are each 0, and W₁ and W₂ are taken together to form an optionally substituted piperazine group.

In some embodiments, m is 1, n, o, p, and q are each 0, W₁ and W₂ are each null, and L is

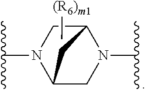

In some embodiments, m is 1, n and p are each 1, o and q are each 0, W₁ and W₂ are each NH, and L is C₃-C₆ cycloalkyl.

In some embodiments, m is 1, n is 1, o, p, and q are each 0, W₁ and W₂ are each NH, and L is C₃-C₆ cycloalkyl.

In some embodiments, m is 1, n, o, p, are each 0, q is 1, W₁ and W₂ are each NH, and L is C₃-C₆ cycloalkyl.

In some embodiments, m is 1, n, o, p, and q are each 0, W₁ is NH, W₂ is null, and L is

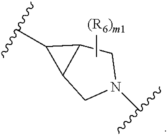

In some embodiments, m is 1, n o, p, and q are each 0, W₁ is null, W₂ is NH, and L is

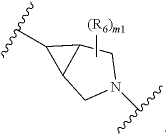

In some embodiments, m is 1, n o, p, and q are each 0, W₁ is NH, W₂ is null, and L is

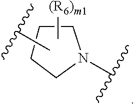

In some embodiments, m is 1, n o, p, and q are each 0, W₁ is null, W₂ is NH, and L is

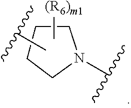

In some embodiments, m is 1, n is 1, o, p, and q are each 0, W₁ is NH, W₂ is null, and L is

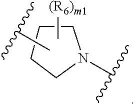

In some embodiments, m is 1, n, o, p, are each 0, q is 1, W₁ is null, W₂ is NH, and L is

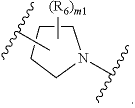

In some embodiments, m is 1, n, o, p, and q are each 0, W₁ is NH, W₂ is null, and L is

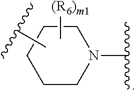

In some embodiments, m is 1, n, o, p, and q are each 0, W₁ is null, W₂ is NH, and L is

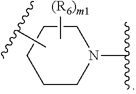

In some embodiments, m is 1, n is 1, o, p, and q are each 0, W₁ is NH, W₂ is null, and L is

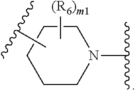

In some embodiments, m is 1, n, o, p, are each 0, q is 1, W₁ is null, W₂ is NH, and L is

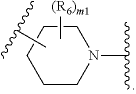

In some embodiments, m is 1, n is 1, o, p, and q are each 0, W₁ is NH, W₂ is null, and L is

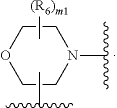

In some embodiments, m is 1, n, o, p, are each 0, q is 1, W₁ is null, W₂ is NH, and L is

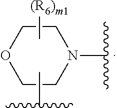

In some embodiments, m is 1, n, o, p, q are each 0, W₁ and W₂ is null, and L is

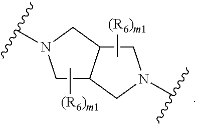

In some embodiments, m is 1, n, o, p, q are each 0, W₁ and W₂ is null, and L is

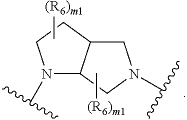

In some embodiments, m is 1, n, o, p, q are each 0, W₁ is NH, W₂ is null, and L is

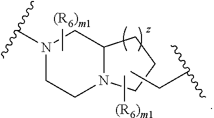

In some embodiments, m is 1, n, o, p, q are each 0, W₁ is null, W₂ is NH, and L is

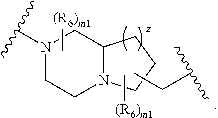

In some embodiments, m is 1, n, o, p, are each 0, q is 1, W₁ and W₂ are each and NH, is null, L is

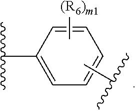

In some embodiments, m is 1, n, o, p, are each 0, q is 1, W₁ and W₂ are each NH, is null, and L is a heteroaryl.

In some of the foregoing embodiments, r is 2, s is 6 and t is 1.

In some of the foregoing embodiments, r is 3, s is 5 and t is 1.

In some of the foregoing embodiments, Z is

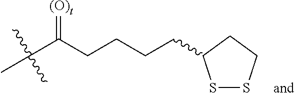

t is 1.

In Formula I, any one or more of H may be substituted with a deuterium. It is also understood in Formula I, that a methyl substituent can be substituted with a C₁-C₆ alkyl.

In other illustrative embodiments, compounds of Formula I are as set forth below:
(4Z,7Z,10Z,13Z,16Z,19Z)-N-(2((E)-6-(4-hydroxy-6-methoxy-7-methyl-3-oxo-1,3- dihydroisobenzofuran-5-yl)-4-methylhex-4-enamido)ethyl)docosa-4,7,10,13,16,19- hexaenamide (I-1),
(4Z,7Z,10Z,13Z,16Z,19Z)-N-(2-(2((E)-6-(4-hydroxy-6-methoxy-7-methyl-3-oxo-1,3- dihydroisobenzofuran-5-yl)-4-methylhex-4-enamido)ethoxy)ethyl)docosa-4,7,10,13,16,19- hexaenamide (I-2),
(4Z,7Z,10Z,13Z,16Z,19Z)-N-(2-((2-((E)-6-(4-hydroxy-6-methoxy-7-methyl-3-oxo-1,3- dihydroisobenzofuran-5-yl)-4-methylhex-4-enamido)ethyl)(methyl)amino)ethyl)docosa- 4,7,10,13,16,19-hexaenamide (I-3),
(4Z,7Z,10Z,13Z,16Z,19Z)-N-(2-((2-((E)-6-(4-hydroxy-6-methoxy-7-methyl-3-oxo-1,3- dihydroisobenzofuran-5-yl)-4-methylhex-4-enamido)ethylamino)ethyl)docosa- 4,7,10,13,16,19-hexaenamide (I-4),
(4Z,7Z,10Z,13Z,16Z,19Z)-N-(2-(2-(2-((E)-6-(4-hydroxy-6-methoxy-7-methyl-3-oxo-1,3- dihydroisobenzofuran-5-yl)-4-methylhex-4-enamido)ethyl)disulfanyl)ethyl)docosa- 4,7,10,13,16,19-hexaenamide (I-5),
methyl 2-(4Z,7Z,10Z,13Z,16Z,19Z)-docosa-4,7,10,13,16,19-hexaenamido-3-(2-((E)-6-(4- hydroxy-6-methoxy-7-methyl-3-oxo-1,3-dihydroisobenzofuran-5-yl)-4-methylhex-4- enamido)propanoyloxy)butanoate (I-6),
methyl 2-(4Z,7Z,10Z,13Z,16Z,19Z)-docosa-4,7,10,13,16,19-hexaenamido-6-((E)-6-(4- hydroxy-6-methoxy-7-methyl-3-oxo-1,3-dihydroisobenzofuran-5-yl)-4-methylhex-4- enamido)hexanoate (I-7),
2-(4Z,7Z,10Z,13Z,16Z,19Z)-docosa-4,7,10,13,16,19-hexaenamido-6-((E)-6-(4-hydroxy-6- methoxy-7-methyl-3-oxo-1,3-dihydroisobenzofuran-5-yl)-4-methylhex-4-enamido)hexanoic acid (I-8),
1,3-dihydroxypropan-2-yl 2-(4Z,7Z,10Z,13Z,16Z,19Z)-docosa-4,7,10,13,16,19- hexaenamido-6-((E)-6-(4-hydroxy-6-methoxy-7-methyl-3-oxo-1,3-dihydroisobenzofuran-5- yl)-4-methylhex-4-enamido)hexanoate (I-9),
(5Z,8Z,11Z,14Z,17Z)-N-(2-((E)-6-(4-hydroxy-6-methoxy-7-methyl-3-oxo-1,3- dihydroisobenzofuran-5-yl)-4-methylhex-4-enamido)ethyl)icosa-5,8,11,14,17-pentaenamide (I-10),
6-(4Z,7Z,10Z,13Z,16Z,19Z)-docosa-4,7,10,13,16,19-hexaenamido-2-((E)-6-(4-hydroxy-6- methoxy-7-methyl-3-oxo-1,3-dihydroisobenzofuran-5-yl)-4-methylhex-4-enamido)hexanoic acid (I-11), and
1,3-dihydroxypropan-2-yl 6-(4Z,7Z,10Z,13Z,16Z,19Z)-docosa-4,7,10,13,16,19- hexaenamido-2-9(E)-6-(4-hydroxy-6-methoxy-7-methyl-3-oxo-1,3-dihydroisobenzofuran-5- yl)-4-methylhex-4-enamido)hexanoate (I-12).
   Methods for using fatty acid mycophenolate derivatives

Described herein are methods of treating and preventing native or transgenic organ, tissue or cellular allograft or xenograft transplant rejection, e.g. for the treatment of recipients of e.g. heart, lung, combined heart-lung, liver, kidney, pancreatic, skin, pancreatic islet cell, neural cell or corneal transplant; including treating and preventing acute rejection; treating and preventing hyperacute rejection, e.g. as associated with xenograft rejection; treating and preventing chronic rejection, e.g. as associated with graft-vessel disease; and treating and preventing graft-versus-host disease, such as following bone marrow transplantation.

The compounds of the invention are useful for reducing, inhibiting or treating restenosis and hyperplasia; hyperproliferative vascular disease and angiogenesis. Because of the immunesuppressant nature of mycophenolate, also described herein are methods of treating and preventing autoimmune diseases, e.g. immune-mediated diseases and inflammatory conditions, in particular inflammatory conditions with an etiology including an immunological component such as arthritis (for instance, rheumatoid arthritis, arthritis chronica progediente and arthritis deformans) and rheumatic diseases. Other immune-mediated diseases for which the fatty acid mycophenolate derivatives may be employed include autoimmune hematological disorders, including, but not limited to hemolytic anaemia, aplastic anaemia, pure red cell anaemia and idiopathic thrombocytopenia), systemic lupus erythematosus, polychondritis, sclerodoma, Wegener granulosis, dermatomyositis, polymyositis, chronic active hepatitis, primary bilary cirrhosis, myasthenia gravis, psoriasis, Steven-Johnson syndrome, pemphigus, idiopathic sprue, inflammatory bowel diseases (including ulcerative colitis and Crohn's disease), endocrine ophthalmopathy, Graves disease, sarcoidosis, multiple sclerosis, juvenile diabetes (diabetes mellitus type 1), non-infection uveitis (antierior and posterior), keratoconjuntivitis sicca nad vernal keratoconjunctivitis, interstitial lung fibrosis, psoriatic arthritis, vasculitis, glomerulonephritides (with and without nephrotic syndrome, e.g. including idiopathic nephrotic syndrome or minimal change nephropathy), IgA nephropathy and juvenile dermatomyositis.

In some embodiments, the subject is administered an effective amount of a fatty acid mycophenolate derivative.

The invention also includes pharmaceutical compositions useful for treating or preventing a metabolic disease, or for inhibiting a metabolic disease, or more than one of these activities. The compositions can be suitable for internal use and comprise an effective amount of a fatty acid mycophenolate derivative and a pharmaceutically acceptable carrier. The fatty acid mycophenolate derivatives are especially useful in that they demonstrate very low peripheral toxicity or no peripheral toxicity.

The fatty acid mycophenolate derivatives can each be administered in amounts that are sufficient to treat or prevent a metabolic disease or prevent the development thereof in subjects.

Administration of the fatty acid mycophenolate derivatives can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a fatty acid mycophenolate derivative and a pharmaceutically acceptable carrier, such as: a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, alginic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the fatty acid mycophenolate derivative is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the fatty acid mycophenolate derivatives.

The fatty acid mycophenolate derivatives can be also formulated as a suppository that can be prepared from fatty emulsions or suspensions; using polyalkylene glycols such as propylene glycol, as the carrier.

The fatty acid mycophenolate derivatives can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in United States Patent No. 5,262,564, the contents of which are herein incorporated by reference in their entirety.

Fatty acid mycophenolate derivatives can also be delivered by the use of monoclonal antibodies as individual carriers to which the fatty acid mycophenolate derivatives are coupled. The fatty acid mycophenolate derivatives can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the fatty acid mycophenolate derivatives can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, fatty acid mycophenolate derivatives are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Compositions can be prepared according to conventional mixing, granulating or coating methods, respectively, and the present pharmaceutical compositions can contain from about 0.1% to about 90%, from about 10% to about 90%, or from about 30% to about 90% of the fatty acid mycophenolate derivative by weight or volume.

The dosage regimen utilizing the fatty acid mycophenolate derivative is selected in accordance with a variety of factors including type, species, age, weight, sex and medical condition of the patient; the severity of the condition to be treated; the route of administration; the renal or hepatic function of the patient; and the particular fatty acid mycophenolate derivative employed. A physician or veterinarian of ordinary skill in the art can readily determine and prescribe the effective amount of the drug required to prevent, counter or arrest the progress of the condition.

Effective dosage amounts of the present invention, when used for the indicated effects, range from about 100 mg to about 5,000 mg of the fatty acid mycophenolate derivative per day. Compositions for in vivo or in vitro use can contain about 100, 150, 250, 500, 750, 1,000, 1,250, 2,500, 3,500, or 5,000 mg of the fatty acid mycophenolate derivative. In one embodiment, the compositions are in the form of a tablet that can be scored. Effective plasma levels of the fatty acid mycophenolate derivative can range from about 5 ng/mL to about 5,000 ng/mL. Appropriate dosages of the fatty acid mycophenolate derivatives can be determined as set forth in Goodman, L. S.; Gilman, A. The Pharmacological Basis of Therapeutics, 5th ed.; MacMillan: New York, 1975, pp. 201-226.

Fatty acid mycophenolate derivatives can be administered in a single daily dose, or the total daily dosage can be administered in divided doses of two, three or four times daily. Furthermore, fatty acid mycophenolate derivatives can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays and gels, wherein the concentration of the fatty acid mycophenolate derivative ranges from about 0.1% to about 15%, w/w or w/v.

### METHODS OF MAKING

Methods for making the fatty acid mycophenolate derivatives

Examples of synthetic pathways useful for making fatty acid mycophenolate derivatives of Formula I are set forth in the Examples below and generalized in Schemes 1-10.

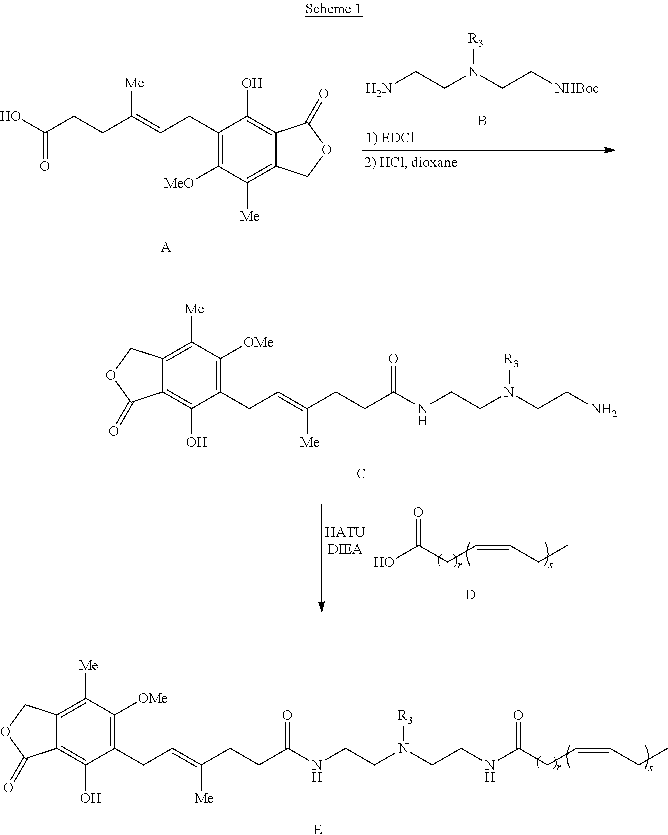

wherein R₃, r, and s are as defined above.

The mono-BOC protected amine of the formula B can be obtained from commercial sources or prepared according to the procedures outlined in Krapcho et al. Synthetic Communications 1990, 20, 2559-2564. The commercially available compound A can be amidated with the amine B using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound C. Activation of compound C with a coupling agent such as HATU in the presence of an amine such as DIEA followed by addition of a fatty acid of formula D affords compounds of the formula E.

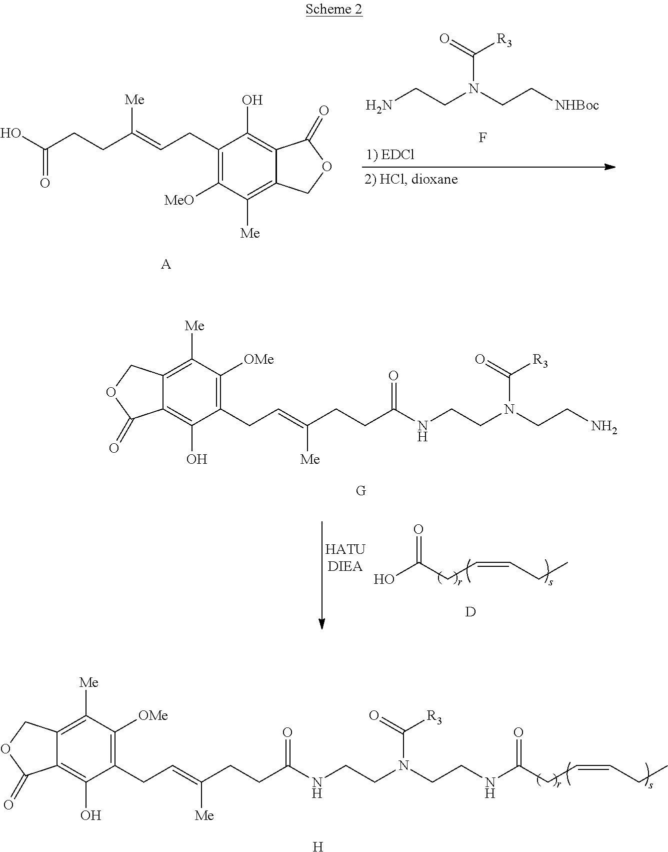

wherein R₃, r, and s are as defined above.

The acylated amine of the formula F can be prepared using the procedures outlined in Andruszkiewicz et al. Synthetic Communications 2008, 38, 905-913. Compound A can be amidated with the amine F using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound G. Activation of compound G with a coupling agent such as HATU in the presence of an amine such as DIEA followed by addition of a fatty acid of formula D affords compounds of the formula H.

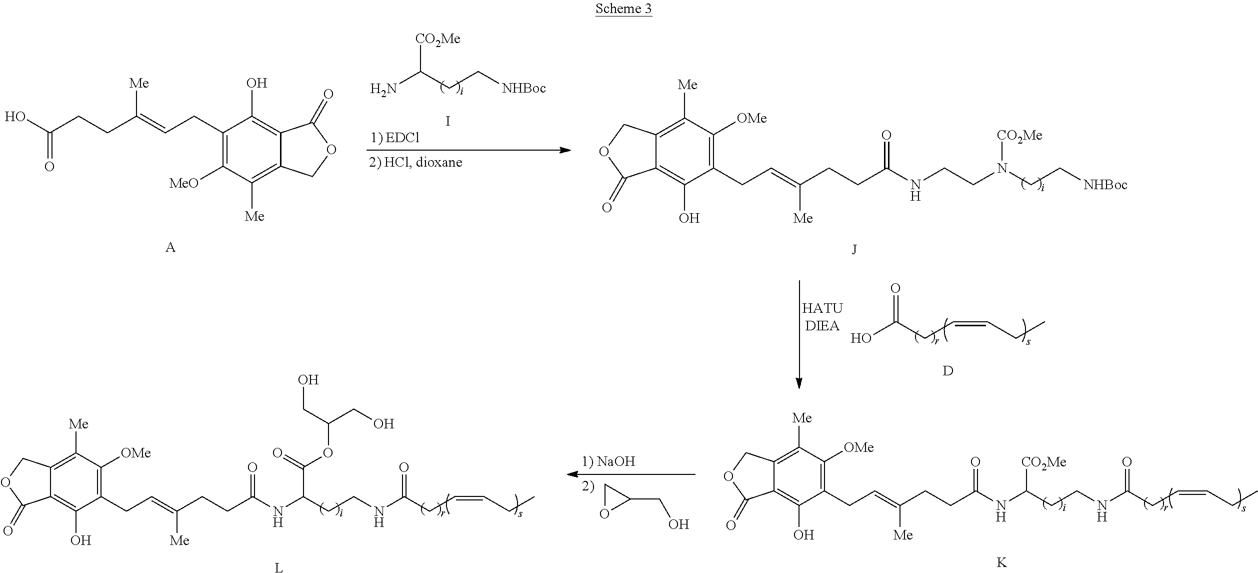

wherein r and s are as defined above.

Compound A can be amidated with the corresponding amine I (where i=0, 1, 2 or 3) using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound J. Activation of compound J with a coupling agent such as HATU in the presence of an amine such as DIEA followed by addition of a fatty acid of formula D affords compounds of the formula K. Hydrolysis of the ester under basic conditions such as NaOH or LiOH produces the corresponding acid, which can be coupled with glycidol to afford compounds of the formula L.

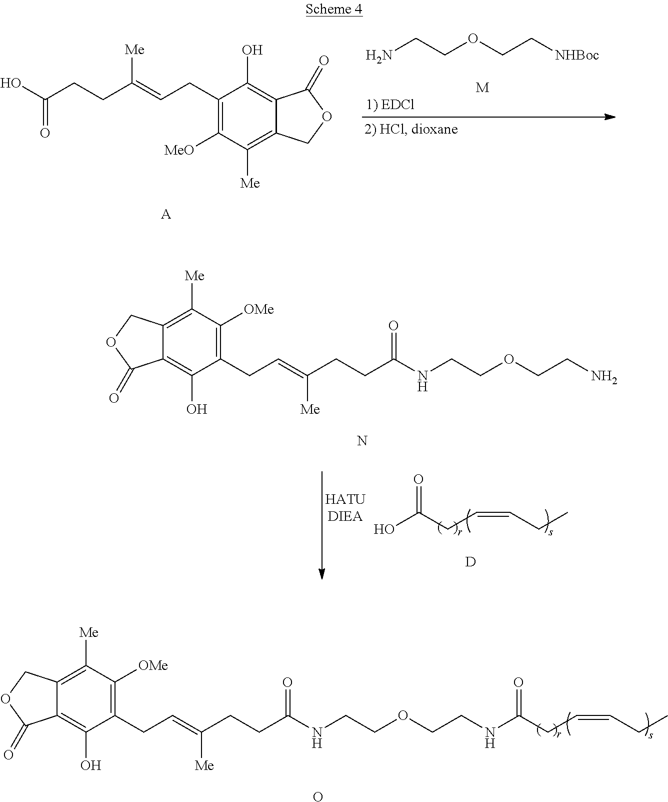

wherein r and s are as defined above.

The amine M can be prepared according to the procedures outlined in Dahan et al. J. Org. Chem. 2007, 72, 2289-2296. Compound A can be coupled with the amine M using a coupling reagent such as DCC, CDI, EDC, or optionally with a tertiary amine base and/or catalyst, e.g., DMAP, followed by deprotection of the BOC group with acids such as TFA or HCl in a solvent such as CH₂Cl₂ or dioxane to produce the coupled compound N. Activation of compound N with a coupling agent such as HATU in the presence of an amine such as DIEA followed by addition of a fatty acid of formula D affords compounds of the formula O.

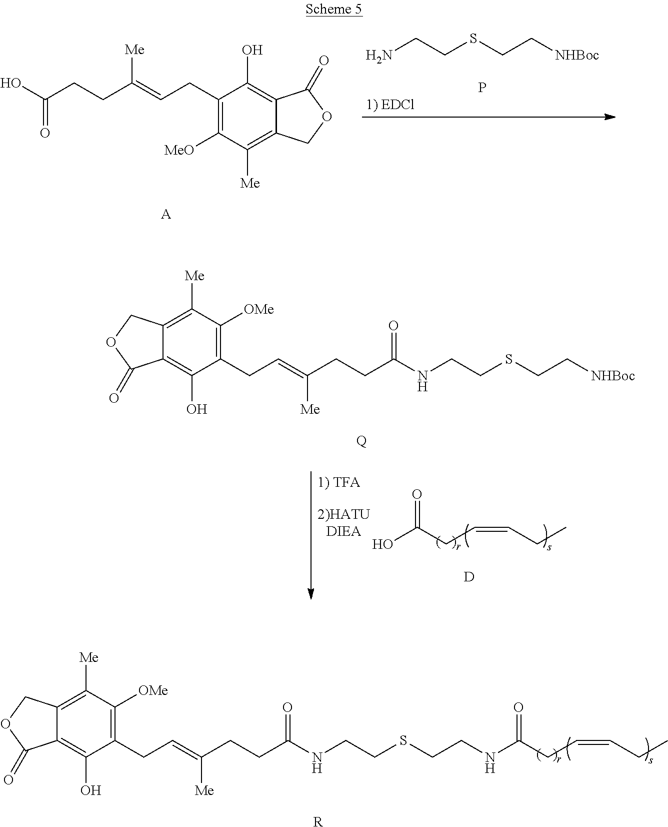

wherein r and s are as defined above.

Compound A can be amidated with the commercially available amine P using a coupling reagent such as DCC, CDI, EDC, or optionally with a tertiary amine base and/or catalyst, e.g., DMAP, to afford compound Q. The BOC group in compound Q can be removed with acids such as TFA or HCl in a solvent such as CH₂Cl₂ or dioxane and the resulting amine can be coupled with a fatty acid of formula D using a coupling agent such as HATU in the presence of an amine such as DIEA to afford compounds of the formula R. To those skilled in the art, the sulfur group in formula Q can be oxidized to the corresponding sulfoxide or sulfone using an oxidizing agent such as H₂O₂ or oxone.

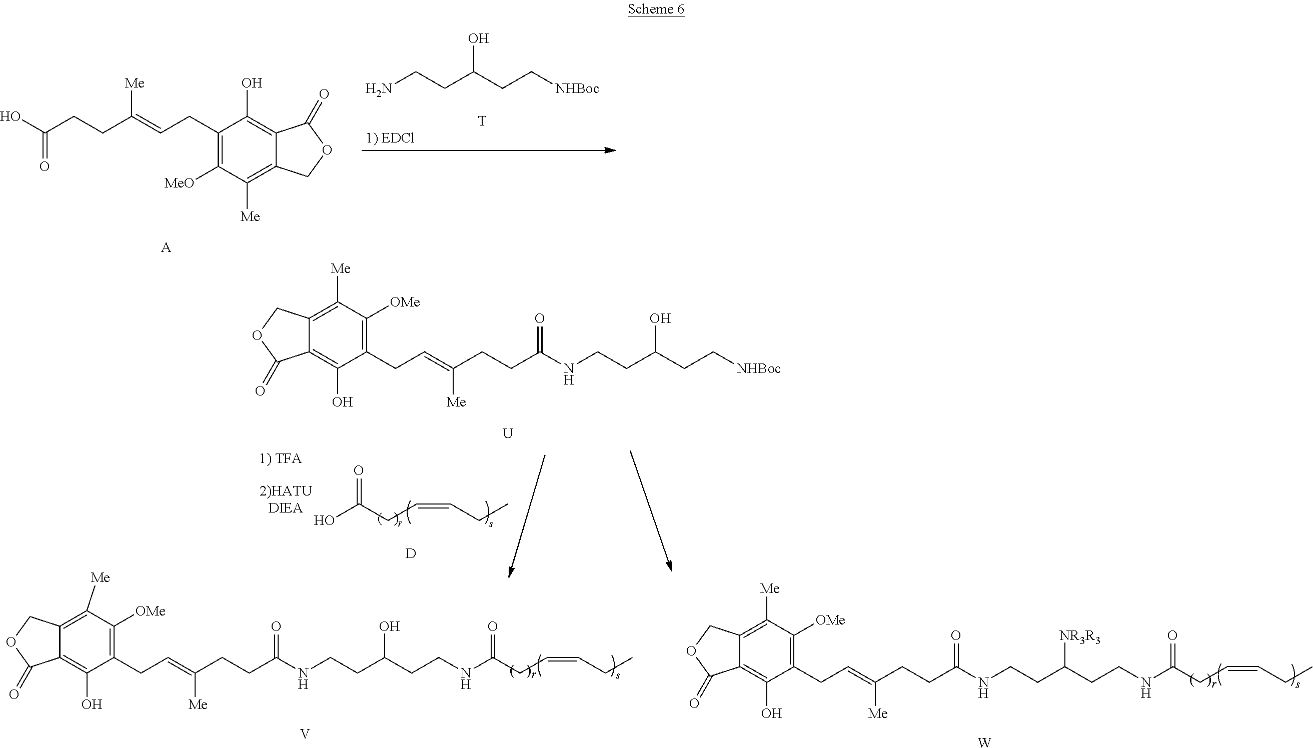

wherein R₃, r, and s are as defined above.

The amine T can be prepared from the commercially available diamine according to the procedures outlined in Dahan et al. J. Org. Chem. 2007, 72, 2289-2296. Compound A can be amidated with the amine T using a coupling reagent such as DCC, CDI, EDC, or optionally with a tertiary amine base and/or catalyst, e.g., DMAP, to afford compound U. The BOC group of compound U can be removed with acids such as TFA or HCl in a solvent such as CH₂Cl₂ or dioxane and the resulting amine can be coupled with a fatty acid of formula D using HATU in the presence of an amine such as DIEA to afford compounds of the formula V. To those skilled in the art, the hydroxyl group in compound U can be further acylated or converted to an amino group by standard mesylation chemistry followed by displacement with sodium azide and hydrogenation over a catalyst such as Pd/C. The amine can be further acylated or alkylated, followed by the removal of the BOC group. The resulting amine can be coupled with a fatty acid of the formula D to afford compounds of the formula W.

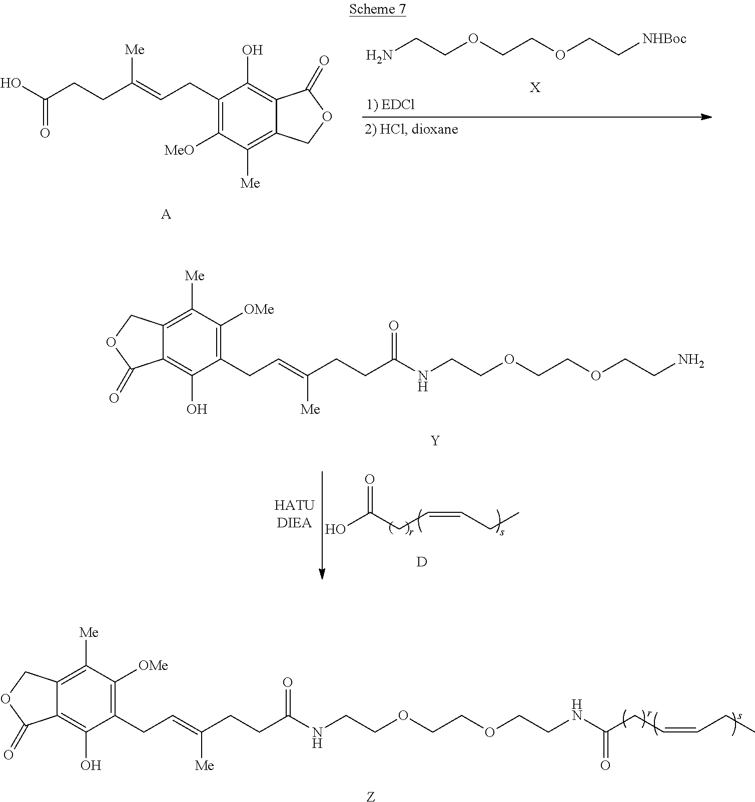

wherein r and s are as defined above.

Compound A can be amidated with the commercially available amine X using a coupling reagent such as DCC, CDI, EDC, optionally with a tertiary amine base and/or catalyst, e.g., DMAP to afford compound Y. The BOC group in compound Y can be removed with acids such as TFA or HCl in a solvent such as CH₂Cl₂ or dioxane. The resulting amine can be coupled with a fatty acid of the formula D using a coupling agent such as HATU in the presence of an amine such as DIEA to afford compounds of the formula Z.

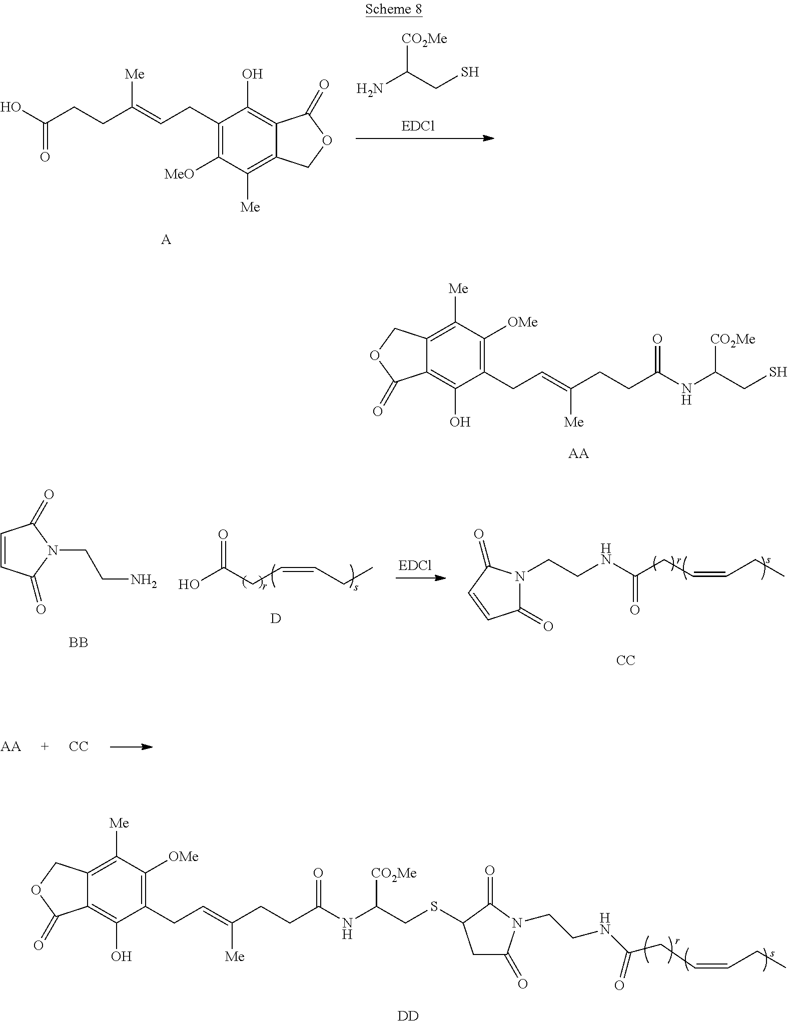

wherein r and s are as defined above.

Compound A can be amidated with the commercially available cysteine methyl ester using a coupling reagent such as DCC, CDI, EDC, or optionally with a tertiary amine base and/or catalyst, e.g., DMAP, to afford compound AA. The commercially available maleimide derivative BB can be coupled with a fatty acid of the formula D using a coupling agent such as HATU or EDCI to afford compounds of the formula CC. Compound AA can be coupled to compounds of the formula CC in a solvent such as acetonitrile to afford compounds of the formula DD.

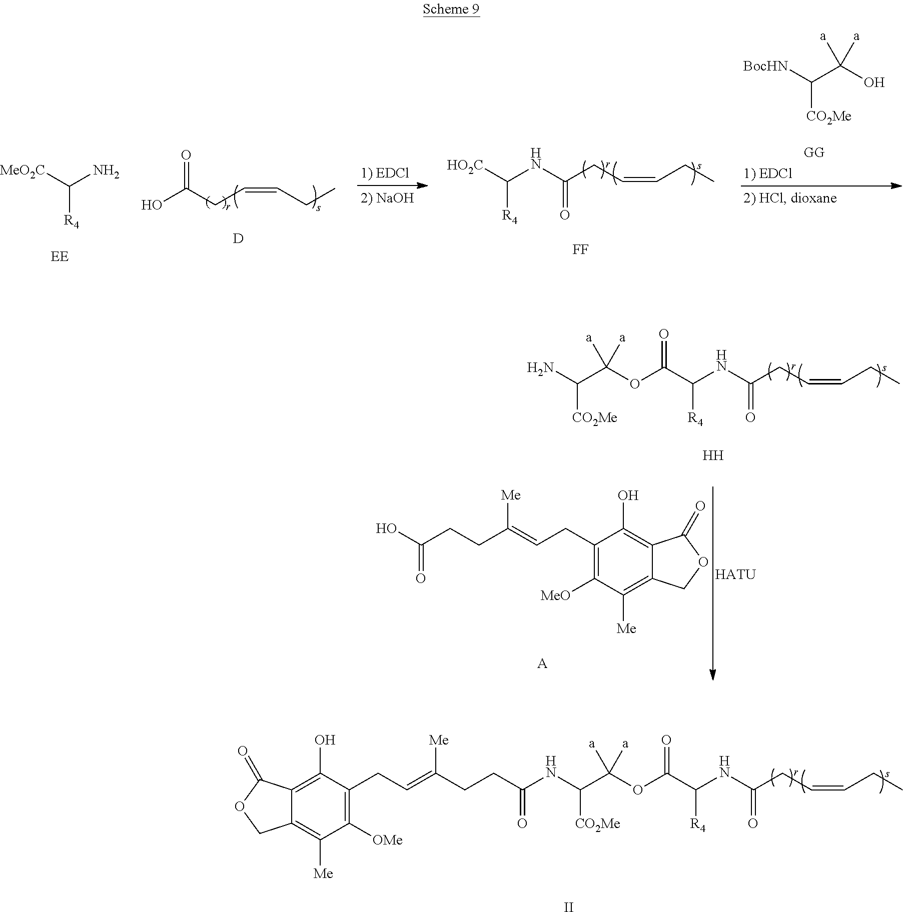

wherein a, R₄, r, and s are as defined above.

The commercially available amino acid esters EE can be coupled with a fatty acid of the formula D using a coupling agent such as EDCI or HATU, followed by alkaline hydrolysis of the methyl ester to afford compounds of the formula FF. Compounds of the formula FF can be coupled with the commercially available BOC-amino acid derivatives GG using a coupling agent such as EDCI or HATU. The BOC group can be removed by treatment with acids such as TFA or HCl to afford compounds of the formula HH which can then be coupled with compound A to afford compounds of the formula II.

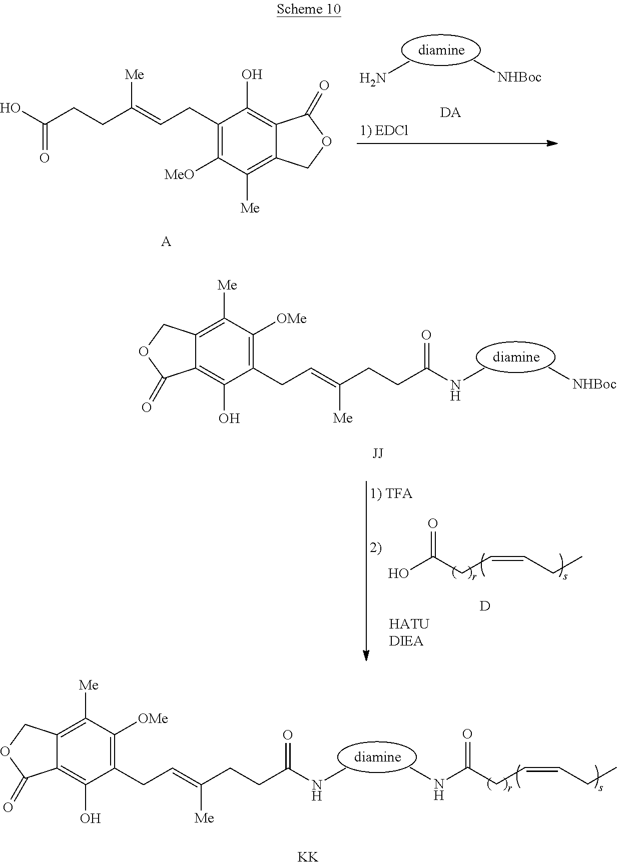

A fatty acid of formula A can be coupled with a BOC-protected diamine of the general formula DA to obtain the BOC-protected amide derivative JJ. After treatment with HCl in dioxane, the resulting amine can be coupled with a fatty acid of the formula A in order to obtain compounds of the formula KK. A variety of BOC-protected diamines are commercially available. The following diamines can be prepared according to the procedures outlined in the corresponding references:

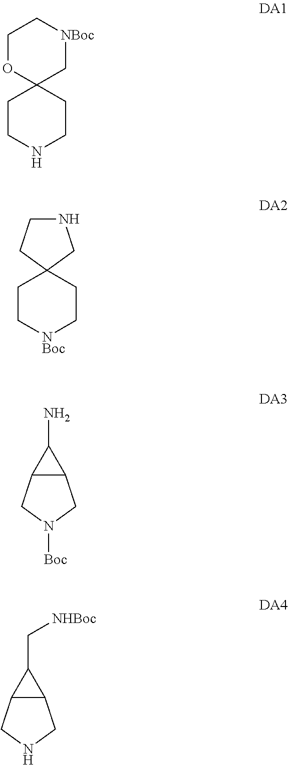

diamine DA1, Stocks et al, Bioorganic and Medicinal Chemistry Letters 2010, p. 7458; diamine DA2, Fritch et al, Bioorganic and Medicinal Chemistry Letters 2010, p. 6375; diamine DA3 and DA4, Moffat et al, J. Med. Chem. 2010, 53, p.8663-8678). To those familiar in the art, detailed procedures to prepare a variety of mono-protected diamines can also be found in the following references: WO 2004092172, WO 2004092171, and WO 2004092173.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

Effect of bis-fatty acid derivatives on IL-1β

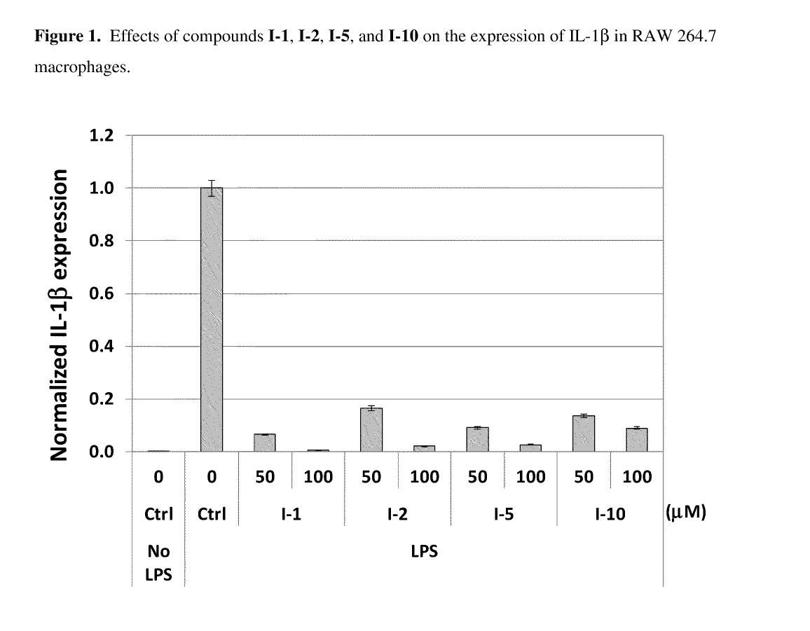

RAW264.7 macrophages were seeded at a density of 100,000 cells/well in a 96-well plate in DMEM supplemented with 10% FBS and Penn/strep. 16 hours later, medium was aspirated and replaced with 90 μL/well of serum-free DMEM. Test compounds were brought up in 100% EtOH to a concentration of 100 mM and then diluted 1:100 in 100% FBS for a stock solution consisting of 1mM compound and 1% EtOH. These stock solutions were then diluted 1:10 in FBS supplemented with 1% EtOH to generate a 100 μM of the test compounds. 10 μL was then added to the RAW246.7 cells to generate final concentrations consisting of 10 μM of the fatty acid mycophenolate conjugate, along with vehicle only control. The compounds were allowed to pre-incubate for 2 hours before stimulation of 100 ng/ml LPS (10 μL of 1 μg/ml LPS was added to each well). Following 3 hours of LPS stimulation, cells were washed once in 1 ×PBS, aspirated dry, and flash frozen in liquid nitrogen. RNA was then isolated and converted to cDNA using the Cells to cDNA kit (Ambion) according to the manufacturer's protocol. IL-1β transcript levels were then measured using Taqman primer/probe assay sets (Applied Biosystems), normalized to GAPDH using the deltaCt method, and the data expressed relative to vehicle only control. FIG. 1 summarizes the effects of compounds I-1, I-2, I-5, and I-10 on the expression of IL-1β in RAW 264.7 mouse macrophages. All four compounds were dosed at 50 μM and 100 μM. Compared to the control vehicle, all four compounds showed a statistically significant decrease in IL-1β expression upon LPS stimulation.

Example 2

The MRLlpr/lpr mouse model for systemic lupus erythematosus (SLE):

MRLlpr/lpr mouse strain spontaneously develops an autoimmune disease resembling human systemic lupus erythematosus (SLE). MRLlpr/lpr can be purchased from known suppliers (one such supplier is Bomholtgard, Ry, Denmark). The degree of albuminuria and haematuria in freshly voided urine can be monitored weekly with standard dipsticks (obtained from Redia-Test, Boehringer). For the study, mice are considered to have developed clinical symptoms of SLE when the albuminuria and haematuria readings show either 3 once, or 2 on at least two separate occasions. A number of clinical parameters are evaluated during a particular study: survival, body weights (taken weekly), albuminuria and haematuria, immunoglobulin levels and anti-(ds)DNA antibodies in serum, frequencies of immunoglobulin-producing B lymphocytes and glomerular deposits immunoglobulin and C3. Serum IgG and IgM antibody levels specific for double-stranded (ds)DNA can be measured by ELISA. IgG1, IgG2a, IgG3 and IgM levels in serum can be determined by single radial immunodiffusion technique according to the protocols outlined in Jonsson et al. Clin. Exp. Immunol. 1999, 116 (3), 534-541. Glomerular deposits of immunoglobulin and complement factor C3 can be visualized by direct immunofluorescence on cryostat sections of kidney specimens using F(ab′)₂ fragments of FITC-conjugated goat anti-mouse immunoglobulin and anti-mouse C3 antibodies (Cappel Labs, Cochraneville, Pa.). The scoring process for glomerular deposits of immunoglobulin and complement factor C3 can be carried out according to the protocols outlined in Jonsson et al. Clin. Exp. Immunol. 1999, 116 (3), 534-541.

For a given study, MRLlpr/lpr female mice can be dosed with either the fatty acid mycophenolate conjugate or the vehicle. The duration of the study can be up to 12 weeks. At the end of the study, mice are sacrificed by cervical dislocation and cervical lymph nodes and spleens removed and weighed. Kidneys are also removed and rapidly frozen for immunohistochemical studies. As a positive control, cyclophosphamide can be administered intraperitoneally at a dose of 1.8 mg/mouse per week, a dose previously used for treatment of lupus-prone mice.

### Compounds

The following non-limiting compound examples serve to illustrate further embodiments of the fatty acid mycophenolate derivatives. It is to be understood that any embodiments listed in the Examples section are embodiments of the fatty acid mycophenolate derivatives and, as such, are suitable for use in the methods and compositions described above.

### Example 3

### Preparation of (4Z,7Z,10Z,13Z,16Z,19Z)-N-(2-((E)-6-(4-hydroxy-6-methoxy-7-methyl- 3-oxo-1,3-dihydroisobenzofuran-5-yl)-4-methylhex-4-enamido)ethyl)docosa- 4,7,10,13,16,19-hexaenamide (I-1)

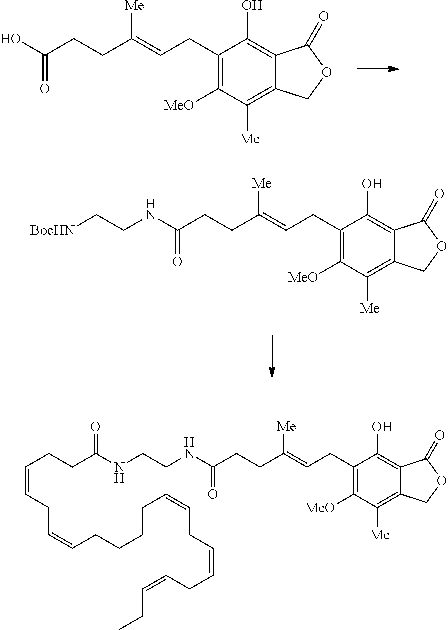

Mycophenolic acid (1.00 g, 3.12 mmol) was taken up in 20 mL of CH₃CN along with tert-butyl 2-aminoethylcarbamate (0.50 g, 3.12 mmol), EDC (660 mg, 3.44 mmol). The resulting reaction mixture was stirred at room temperature for 18. It was then diluted with EtOAc (100 mL) and washed with brine, dried (Na₂SO₄) and concentrated under reduced pressure. Purification by chromatography (95% CH₂Cl₂, 5% MeOH) afforded 1.2 g of (E)-tert-butyl 2-(6-(4-hydroxy-6-methoxy-7-methyl-3-oxo-1,3-dihydroisobenzofuran-5-yl)-4-methylhex-4-enamido)ethylcarbamate. This material was treated with 10 mL of 4 M HCl in dioxane and allowed to stir at room temperature for 2 h. The reaction mixture was diluted with EtOAc (30 mL) and concentrated under reduced pressure to afford the HCl salt of (E)- N-(2-aminoethyl)-6-(4-hydroxy-6-methoxy-7-methyl-3-oxo-1,3-dihydroisobenzofuran-5-yl)- 4-methylhex-4-enamide For the final amide coupling, the HCl salt of (E)-N-(2-aminoethyl)- 6-(4-hydroxy-6-methoxy-7-methyl-3-oxo-1,3-dihydroisobenzofuran-5-yl)-4-methylhex-4- enamide (1.30 mmol) was taken up in 5 mL of CH₃CN along with (4Z,7Z,10Z,13Z,16Z,19Z)-docosa-4,7,10,13,16,19-hexaenoic acid (DHA, 426 mg, 1.30 mmol), HATU (543 mg, 1.43 mmol) and DIEA (680 2.90 mmol). The resulting reaction mixture was stirred at room temperature for 2 h. It was then diluted with EtOAc (30 mL) and washed with brine. The organic layer was dried (Na₂SO₄) and concentrated under reduced pressure. Purification by chromatography (EtOAc/pentane 1:1) afforded 300 mg of (4Z,7Z,10Z,13Z,16Z,19Z)-N-(2-((E)-6-(4-hydroxy-6-methoxy-7-methyl-3-oxo-1,3- dihydroisobenzofuran-5-yl)-4-methylhex-4-enamido)ethyl)docosa-4,7,10,13,16,19- hexaenamide (34% yield). MS (EI) calcd for C₄₁H₅₆N₂O₆: 672.41; found 673 (M+1).

### Example 4

### Preparation of (5Z,8Z,11Z,14Z,17Z)-N-(2-((E)-6-(4-hydroxy-6-methoxy-7-methyl-3-oxo- 1,3-dihydroisobenzofuran-5-yl)-4-methylhex-4-enamido)ethyl)icosa-5,8,11,14,17- pentaenamide (I-10)

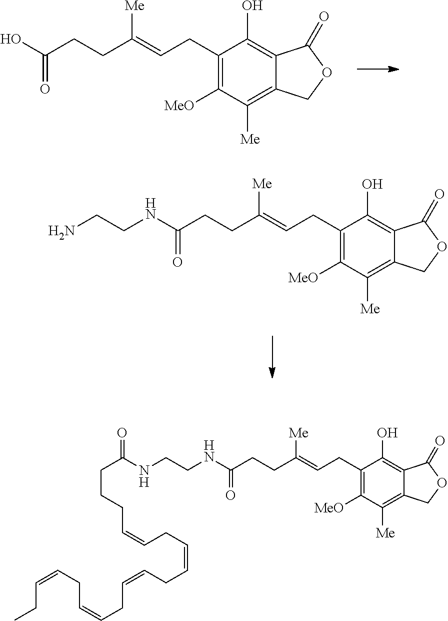

The same experimental procedure outlined above in the synthesis of (4Z,7Z,10Z,13Z,16Z,19Z)-N-(2-((E)-6-(4-hydroxy-6-methoxy-7-methyl-3-oxo-1,3- dihydroisobenzofuran-5-yl)-4-methylhex-4-enamido)ethyl)docosa-4,7,10,13,16,19- hexaenamide was used, substituting (5Z,8Z,11Z,14Z,17Z)-eicosa-5,8,11,14,17-pentaenoic acid (EPA) for DHA. MS (EI) calcd for C₃₉H₅₄N₂O₆: 646.4; found 647 (M+1).

### Example 5

### Preparation of (4Z,7Z,10Z,13Z,16Z,19Z)-N-(2-(2-(2-((E)-6-(4-hydroxy-6-methoxy-7- methyl-3-oxo-1,3-dihydroisobenzofuran-5-yl)-4-methylhex-4- enamido)ethyl)disulfanyl)ethyl)docosa-4,7,10,13,16,19-hexaenamide (I-4)

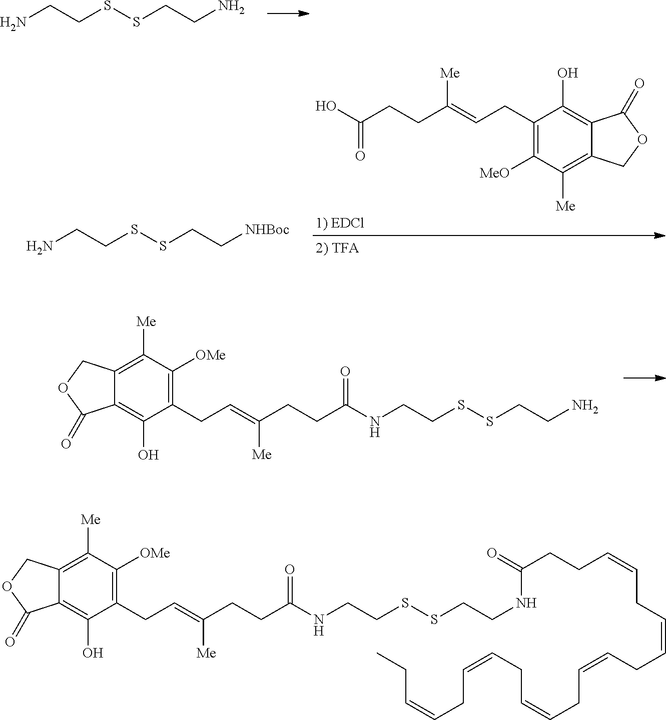

Cystamine dihydrochloride (1.0 g, 4.44 mmol) was dissolved in MeOH (50 mL). Triethylamine (1.85 mL, 3 eq) was added at room temperature, followed by dropwise addition of Boc₂O (0.97 g, 4.44 mmol) as a solution in MeOH (5 mL). The resulting reaction mixture was stirred at room temperature for 3 h. It was then concentrated under reduced pressure and the resulting residue was taken up in 1M aqueous NaH₂PO₄ (20 mL). The aqueous layer is washed with a 1:1 solution of pentane/EtOAc (10 mL), basified to pH 9 with 1M aqueous NaOH, and extracted with EtOAc. The combined organic layers were washed with brine, dried over Na₂SO₄, filtered and concentrated under reduced pressure to afford tert-butyl 2-(2-(2-aminoethyl)disulfanyl)ethylcarbamate (500 mg, 44%).

tert-Butyl 2-(2-(2-aminoethyl)disulfanyl)ethylcarbamate (1 2 mmol) was taken up in CH₃CN (5 mL) along with mycophenolic acid (1.2 mmol) and EDCI (1.3 mmol). The resulting reaction mixture was stirred at room temperature for 18 h and diluted with EtOAc. The organic layer was washed with saturated aqueous NaHCO₃, brine, dried over Na₂SO₄, filtered and concentrated under reduced pressure. The resulting residue was purified by silica gel chromatography (CH₂Cl₂) to afford (E)-tert-butyl 2-(2-(2-(6-(4-hydroxy-6-methoxy-7- methyl-3-oxo-1,3-dihydroisobenzofuran-5-yl)-4-methylhex-4- enamido)ethyl)disulfanyl)ethylcarbamate.

(E)-tert-butyl 2-(2-(2-(6-(4-hydroxy-6-methoxy-7-methyl-3-oxo-1,3- dihydroisobenzofuran-5-yl)-4-methylhex-4-enamido)ethyl)disulfanyl)ethylcarbamate (0.56 mmol) was taken up in 5 mL of 4 M HCl in dioxane and allowed to stand at room temperature for 2 h. The resulting reaction mixture is concentrated under reduced pressure to afford the HCl salt of (E)-N-(2-(2-(2-aminoethyl)disulfanyl)ethyl)-6-(4-hydroxy-6-methoxy- 7-methyl-3-oxo-1,3-dihydroisobenzofuran-5-yl)-4-methylhex-4-enamide. This material is taken up in CH₃CN (5 mL) along with (4Z,7Z,10Z,13Z,16Z,19Z)-docosa-4,7,10,13,16,19- hexaenoic acid (DHA, 183 mg, 0.56 mmol), HATU (234 mg, 0.62 mmol) and DIEA (290 1.7 mmol). The resulting reaction mixture was stirred at room temperature for 2 h. It was then diluted with EtOAc and washed successively with saturated aqueous NaHCO₃ and brine. The organic layer was dried over Na₂SO₄, filtered and concentrated under reduced pressure. Purification by silica gel chromatography (5% MeOH-CH₂Cl₂) afforded 200 mg of (4Z,7Z,10Z,13Z,16Z,19Z)-N-(2-(2-(2-9(E)-6-(4-hydroxy-6-methoxy-7-methyl-3-oxo-1,3- dihydroisobenzofuran-5-yl)-4-methylhex-4-enamido)ethyl)disulfanyl)ethyl)docosa- 4,7,10,13,16,19-hexaenamide (47% yield). MS (EI) calcd for C₄₃H₆₀N₂O₆S₂: 764.39; found 765 (M+1).

### Example 6

### Preparation of (4Z,7Z,10Z,13Z,16Z,19Z)-N-(2-(2-((E)-6-(4-hydroxy-6-methoxy-7- methyl-3-oxo-1,3-dihydroisobenzofuran-5-yl)-4-methylhex-4- enamido)ethoxy)ethyl)docosa-4,7,10,13,16,19-hexaenamide (I-2)

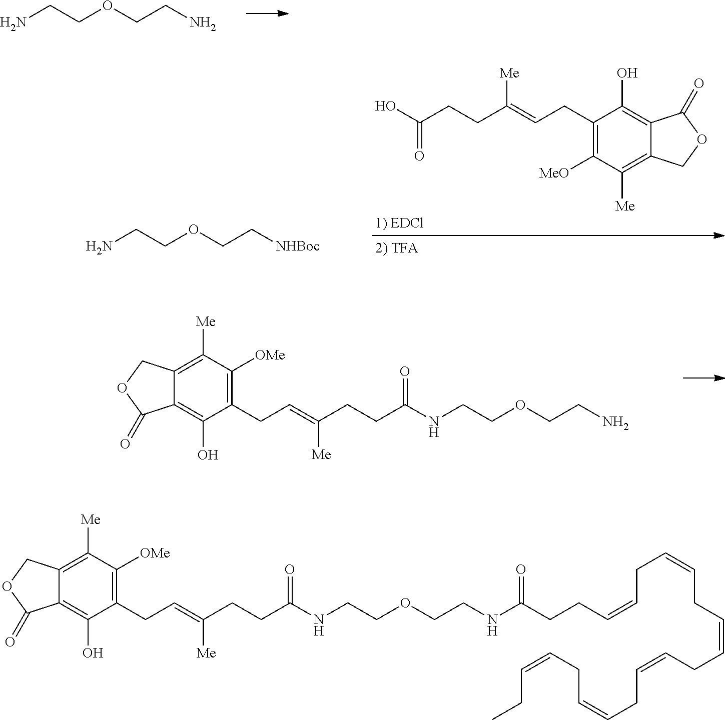

In a typical run, sodium hydroxide (400 mg, 10 mmol) was dissolved in MeOH (70 mL) and 2-(2-aminoethoxy)ethanamine dihydrochloride (1.0 g, 5.65 mmol) was added. The resulting reaction mixture was stirred at room temperature for 30 min A solution containing Boc₂O (740 mg, 3.40 mmol) in THF (15 mL) was then added dropwise, at room temperature, over a period of 15 min The resulting reaction mixture was stirred at room temperature for 18 h, and then concentrated under reduced pressure. The resulting residue was taken up in CH₂Cl₂ (200 mL) and stirred vigorously at room temperature for 4 h. The mixture was filtered and the filtrate was concentrated under reduced pressure to afford tert-butyl 2-(2-aminoethoxy)ethylcarbamate (850 mg, 74%).

tert-Butyl 2-(2-aminoethoxy)ethylcarbamate (1.47 mmol) was then taken up in CH₃CN (10 mL) along with mycophenolic acid (470 mg, 1.47 mmol) and EDCI (310 mg, 1.62 mmol). The resulting reaction mixture was stirred at room temperature for 18 h. It is then diluted with EtOAc (50 mL), washed with saturated aqueous NaHCO₃, brine, dried over Na₂SO₄, filtered and concentrated under reduced pressure. The resulting residue was purified by silica gel chromatography (9:1 CH₂Cl₂/MeOH) to afford 620 mg of (E)-tert-butyl 2-(2-(6- (4-hydroxy-6-methoxy-7-methyl-3-oxo-1,3-dihydroisobenzofuran-5-yl)-4-methylhex-4- enamido)ethoxy)ethylcarbamate (83% yield).

(E)-tert-Butyl 2-(2-(6-(4-hydroxy-6-methoxy-7-methyl-3-oxo-1,3- dihydroisobenzofuran-5-yl)-4-methylhex-4-enamido)ethoxy)ethylcarbamate (620 mg, 1.22 mmol) was taken up in 10 mL of 4 M HC1 in dioxane and allowed to stir at room temperature for 2 h. The resulting reaction mixture was concentrated under reduced pressure to afford the HCl salt of (E)-N-(2-(2-aminoethoxy)ethyl)-6-(4-hydroxy-6-methoxy-7-methyl-3-oxo-1,3- dihydroisobenzofuran-5-yl)-4-methylhex-4-enamide This material was taken up in CH₃CN (10 mL) along with (4Z,7Z,10Z,13Z,16Z,19Z)-docosa-4,7,10,13,16,19-hexaenoic acid (400 mg, 1.22 mmol), HATU (510 mg, 1.34 mmol) and DIEA (640 μL, 3.66 mmol). The resulting reaction mixture was stirred at room temperature for 2 h. It was then diluted with EtOAc and washed successively with saturated aqueous NaHCO₃ and brine. The organic layer was dried over Na₂SO₄, filtered and concentrated under reduced pressure. Purification by silica gel chromatography (5% MeOH in CH₂Cl₂) afforded 600 mg of (4Z,7Z,10Z,13Z,16Z,19Z)-N-(2- (2-((E)-6-(4-hydroxy-6-methoxy-7-methyl-3-oxo-1,3-dihydroisobenzofuran-5-yl)-4- methylhex-4-enamido)ethoxy)ethyl)docosa-4,7,10,13,16,19-hexaenamide (68% yield). MS (EI) calcd for C₄₃H₆₀N₂O₇: 716.44; found 717 (M+1).

### Example 7

### Preparation of (4Z,7Z,10Z,13Z,16Z,19Z)-N-(2-((2-((E)-6-(4-hydroxy-6-methoxy-7- methyl-3-oxo-1,3-dihydroisobenzofuran-5-yl)-4-methylhex-4- enamido)ethyl)(methyl)amino)ethyl)docosa-4,7,10,13,16,19-hexaenamide (I-3)

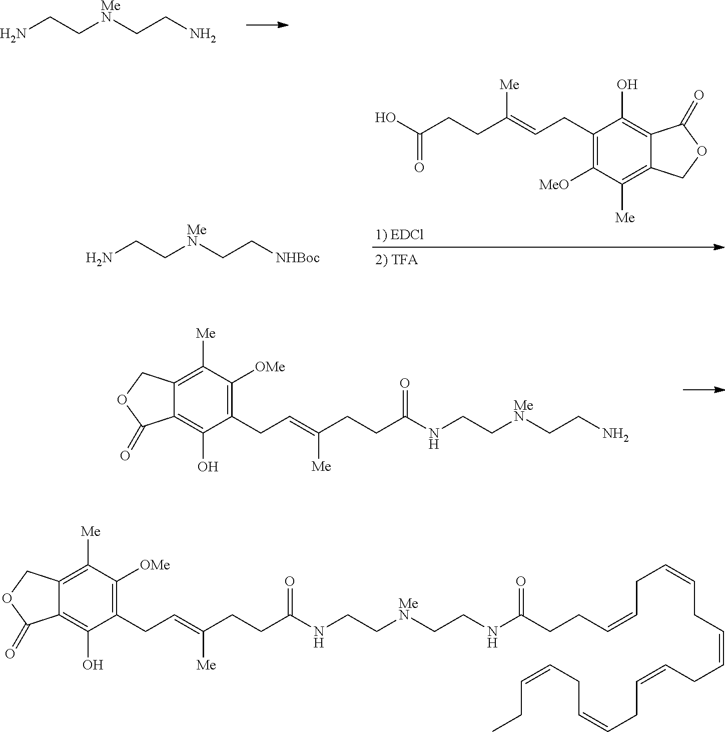

N1-(2-Aminoethyl)-N1-methylethane-1,2-diamine (5.0 g, 42.7 mmol) was dissolved in 100 mL of CH₂Cl₂ and cooled to 0° C. A solution of di-tert-butylcarbonate (0.93 g, 4.27 mmol) in CH₂Cl₂ (10 mL) was then added dropwise at 0° C. over a period of 15 min The resulting reaction mixture was stirred at 0° C. for 30 min and then warmed to room temperature. After stirring at room temperature for 2 h, the reaction mixture was diluted with CH₂Cl₂ (100 mL). The organic layer was washed with brine (3×25 mL), dried (Na₂SO₄) and concentrated under reduced pressure to afford 1.1 g of tert-butyl 2-((2-aminoethyl)(methyl)amino)ethylcarbamate.

tert-butyl 2-((2-aminoethyl)(methyl)amino)ethylcarbamate (200 mg, 0.922 mmol) was taken up in 5 mL of CH₃CN along with mycophenolic acid (295 mg, 0.922 mmol) and EDC (194 mg, 1.01 mmol). The resulting reaction mixture was stirred at room temperature for 18 h and diluted with EtOAc. The organic layer was washed with brine, dried (Na₂SO₄) and concentrated under reduced pressure. Purification by chromatography (95% CH₂Cl₂, 5% MeOH) afforded 400 mg of (E)-tert-butyl 2-((2-(6-(4-hydroxy-6-methoxy- 7-methyl-3-oxo-1,3-dihydroisobenzofuran-5-yl)-4-methylhex-4- enamido)ethyl)(methyl)amino)ethylcarbamate (84% yield). This material was taken up in 10 mL of 4 M HCl in dioxane and allowed to stir at room temperature for 2 h. The mixture was diluted with EtOAc (30 mL) and concentrated under reduced pressure to afford the HC1 salt of (E)-N-(2-((2-aminoethyl)(methyl)amino)ethyl)-6-(4-hydroxy-6-methoxy-7-methyl-3-oxo- 1,3-dihydroisobenzofuran-5-yl)-4-methylhex-4-enamide. This material was then taken up in 10 mL of CH₃CN along with (4Z,7Z,10Z,13Z,16Z,19Z)-docosa-4,7,10,13,16,19-hexaenoic acid (DHA, 252 mg, 0.77 mmol), HATU (322 mg, 0.85 mmol) and DIEA (540 3.1 mmol). The resulting reaction mixture was stirred at room temperature for 2 h and diluted with EtOAc (50 mL). The organic layer was washed with brine, dried (Na₂SO₄) and concentrated under reduced pressure. Purification by chromatography (95% CH₂Cl₂, 5% MeOH) afforded 300 mg of (4Z,7Z,10Z,13Z,16Z,19Z)-N-(2-((2-((E)-6-(4-hydroxy-6- methoxy-7-methyl-3-oxo-1,3-dihydroisobenzofuran-5-yl)-4-methylhex-4- enamido)ethyl)(methyl)amino)ethyl)docosa-4,7,10,13,16,19-hexaenamide (53% yield). MS (EI) calcd for C₄₄H₆₃N₃O₆: 729.47; found 730 (M+1).

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A molecular conjugate comprising a mycophenolate and a fatty acid covalently linked, wherein the fatty acid is selected from omega-3 fatty acids, fatty acids metabolized in vivo into omega-3 fatty acids, or lipoic acid, and the conjugate is capable of hydrolysis to produce free mycophenolate and free fatty acid.

2. A compound of Formula I: 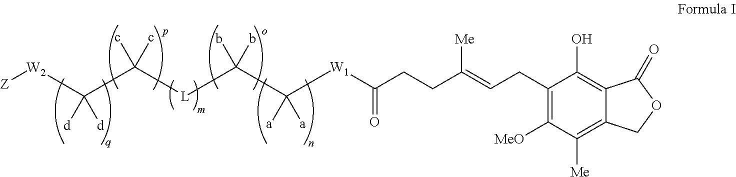
or a pharmaceutically acceptable salt, hydrate, solvate, prodrug, enantiomer, or stereoisomer thereof; wherein
W₁ and W₂ are each independently null, O, S, NH, NR, or W₁ and W₂ can be taken together can form an imidazolidine or piperazine group,
each a, b, c, and d is independently —H, -D, —CH₃, —OCH₃, —OCH₂CH₃, —C(O)OR, —O-Z, or benzyl, or two of a, b, c, and d can be taken together, along with the single carbon to which they are bound, to form a cycloalkyl or heterocycle;
each n, o, p, and q is independently 0, 1 or 2;
L is independently null, —O—, —S—, —S(O)—, —S(O)₂—, —S—S—, —(C₁-C₆alkyl)-, —(C₃-C₆cycloalkyl)-, a heterocycle, a heteroaryl, 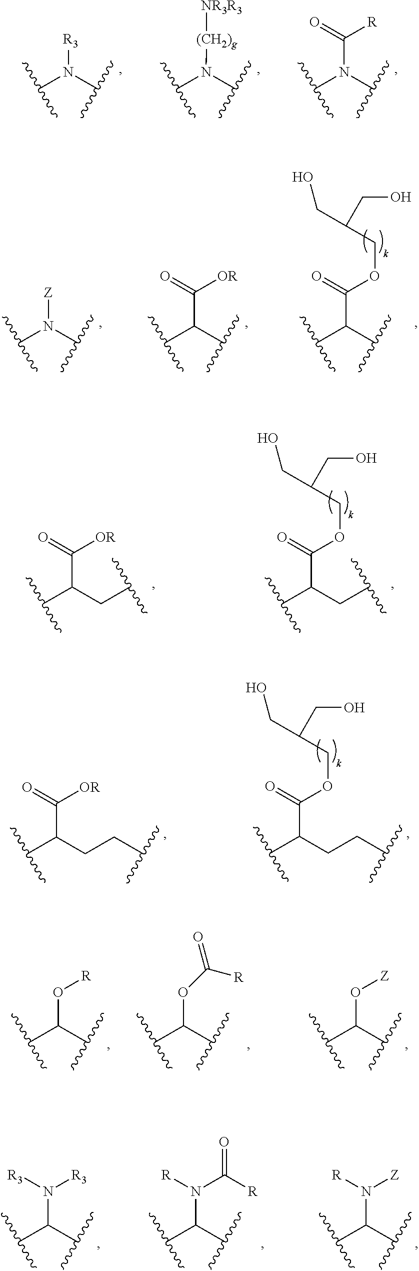 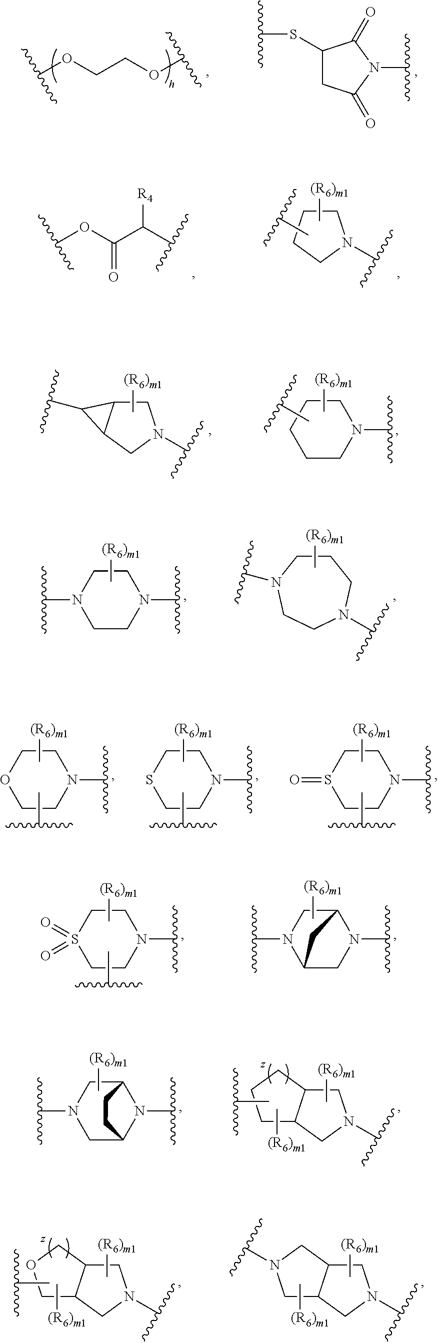 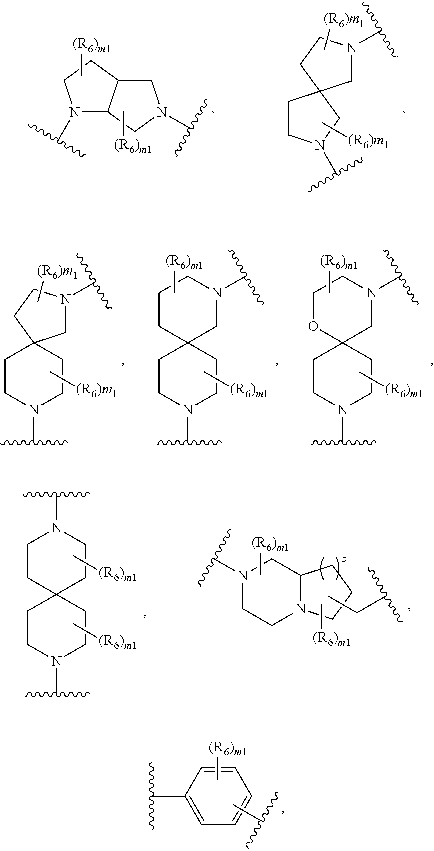
wherein the representation of L is not limited directionally left to right as is depicted, rather either the left side or the right side of L can be bound to the W₁ side of the compound of Formula I;
R₆ is independently -—H, -D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl;
each g is independently 2, 3 or 4;
each h is independently 1, 2, 3 or 4;
m is 0, 1, 2, or 3; if m is more than 1, then L can be the same or different;
m1 is 0, 1, 2 or 3;
k is 0, 1, 2, or 3;
z is 1, 2, or 3;
each R₃ is independently H or C₁-C₆ alkyl that can be optionally substituted with either O or N and in NR₃R₃, both R₃ when taken together with the nitrogen to which they are attached can form a heterocyclic ring such as a pyrrolidine, piperidine, morpholine, piperazine or pyrrole;
each R₄ is independently e, H or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each Z is independently —H, 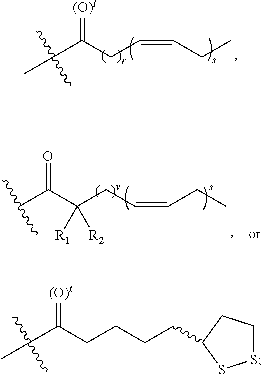
with the proviso that there is at least one 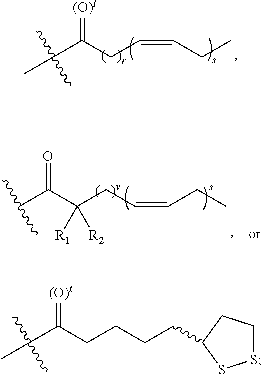
in the compound;
each r is independently 2, 3, or 7;
each s is independently 3, 5, or 6;
each t is independently 0 or 1;
each v is independently 1, 2, or 6;
R₁ and R₂ are each independently hydrogen, deuterium, —C₁-C₄ alkyl, -halogen, —OH, —C(O)C₁-C₄ alkyl, —O-aryl, —O-benzyl, —OC(O)C₁-C₄ alkyl, —C₁-C₃ alkene, —C₁-C₃ alkyne, —C(O)C₁-C₄ alkyl, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, —S(O)₂C₁-C₃ alkyl; and
each R is independently —H, —C₁-C₃ alkyl, or straight or branched C₁-C₄ alkyl optionally substituted with OH, or halogen;
provided that
when m, n, o, p, and q are each 0, W₁ and W₂ are each null, and Z is 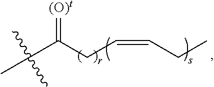
then t must be 0; and
when each of m, n, o, p, and q is 0, and W₁ and W₂ are each null, then Z must not be

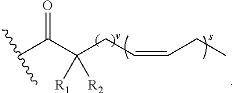

3. A pharmaceutical composition comprising a molecular conjugate of claim 1 and a pharmaceutically acceptable carrier.

4. A pharmaceutical composition comprising a compound of claim 2 and a pharmaceutically acceptable carrier.

5. A method for preventing or treating a disease or disorder wherein the disease or disorder is selected from the group consisting of preventing and reducing the frequency and severity of acute rejection episodes in organ transplants; reducing, inhibiting or treating restenosis and hyperplasia; hyperproliferative vascular disease;
angiogenesis; systemic lupus erythematosus; psoriasis, and multiple sclerosis, the method comprising administering to a patient in need thereof an effective amount of a molecular conjugate of claim 1.

6. A method for preventing or treating a disease or disorder wherein the disease or disorder is selected from the group consisting of preventing and reducing the frequency and severity of acute rejection episodes in organ transplants; reducing, inhibiting or treating restenosis and hyperplasia; hyperproliferative vascular disease;
angiogenesis; systemic lupus erythematosus; psoriasis, and multiple sclerosis, the method comprising administering to a patient in need thereof an effective amount of a compound of claim 2.

7. The method of claim 6, wherein the disease or disorder is systemic lupus erythematosus.

8. The method of claim 6, wherein the disease or disorder is multiple sclerosis.

9. The method of claim 6, wherein the disease or disorder is psoriasis.
